(19) United States
(12) Patent Application Publication
(10) Pub. No.: US 2006/0172943 A1
(43) Pub. Date: Aug. 3, 2006

(54) Restoring vascular function

(76) Inventors: Jay Edelberg, New York, NY (US); Victoria Ballard, New York, NY (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/337,981

(22) Filed: Jan. 23, 2006

Related U.S. Application Data
(63) Continuation of application No. PCT/US04/23321, filed on Jul. 20, 2004.
(60) Provisional application No. 60/489,715, filed on Jul. 24, 2003.

(51) Int. Cl. A61K 38/17

(52) U.S. Cl. 514/12

(57) ABSTRACT

The invention relates to Tenascin-C and peptides that bind thereto. According to the invention, Tenascin-C or peptides and antibodies that bind thereto can be used to treat or prevent vascular diseases, either alone or in combination with therapeutic agents or cells that have cardioplastic potential.

### CROSS-REFERENCE TO RELATED APPLICATIONS

This application is a Continuation Under 35 U.S.C. §1.111a) of International Application No. PCT/US2004/023321 filed Jul. 20, 2004 and published in English as WO 2005/009366 A3 on Feb. 3, 2005,which claims the benefit under 35 U.S.C. §119(e) of U.S. Provisional Application No. 60/489,715 filed Jul. 24, 2003, which applications are incorporated herein by reference.

### GOVERNMENT FUNDING

The invention described in this application was made with funds from the National Institute of Health, Grant Number R01 AG20918-01. The United States government has certain rights in the invention.

### FIELD OF THE INVENTION

The invention relates to compositions of Tenascin-C and methods for using those compositions to treat and prevent vascular conditions, including cardiovascular disease. In another embodiment, the invention relates to peptide that bind Tenascin-C and that can home to bone marrow and cardiac microvascular tissues. The invention further relates to methods for using these compositions and peptides for treating vascular diseases, including cardiovascular disease and atherosclerosis.

### BACKGROUND OF THE INVENTION

In the United States and Western Europe, cardiovascular disease and its associated maladies, dysfunctions and complications are a principal cause of disability and the chief cause of death. One entity significantly contributing to this pathophysiologic process is cardiovascular disease, which has been generally recognized as the leading health care problem both with respect to mortality and health care costs. The American Heart Association estimates that 953,110 persons died of cardiovascular diseases in 1997 (41.2 percent of all deaths), more than the number of mortality for cancer (539,377), accidents (95,644) and HIV (16,516) combined. Furthermore, the American Heart Association calculates that close to a quarter of the population of the United States suffers from one or more forms of cardiovascular disease. American Heart Assoc., 2000, www.americanheart.org/Heart_and_Stroke_A_Z_Guide/cvds.html. Moreover, the medical costs associated with coronary heart disease are estimated at $95 billion dollars a year. Gonzalez & Kannewurf, 55 (19) American Journal of Health-System Pharmacy S4-7 (Supp. 1, 1998).

New methods and compositions are clearly needed for treating and preventing cardiovascular diseases.

### SUMMARY OF THE INVENTION

According to the invention, Tenascin-C protein is expressed in the normal adult heart, by a sub-population of endothelial cells. As illustrated herein, cardiac endothelial cells grown on Tenascin-C display diminished spreading and delayed adhesion compared to those grown on collagen. Moreover, Tenascin-C is expressed in bone marrow, as well as in the heart at sites of wound healing after myocardial infarction. According to the invention, Tenascin-C acts to maintain cardiac endothelial cells, and possibly bone-marrow derived endothelial progenitor cells (EPCs), in an undifferentiated state with diminished cell adhesion and an increased tendency to migrate, as a precursor to vascular remodeling. Moreover, Tenascin-C expression is upregulated in cardiac endothelial cells within 24 hours of administration of platelet derived growth factor (PDGF), both in vitro and in vivo, suggesting that Tenascin-C may act as a downstream mediator of PDGF signaling in cardiac angiogenic pathways. Thus, as described herein, Tenascin-C can promote cardiac angiogenesis and cardioprotection via local and/or systemic mechanisms.

In one aspect, the invention is directed to compositions formulated for delivery to a site of vascular injury or vascular disease, wherein the compositions contain a therapeutically effective amount of Tenascin-C and a pharmaceutically acceptable carrier. In some embodiments, a therapeutically effective amount of Tenascin-C is an amount that can modulate migration of cardiac endothelial cells. Another aspect of the invention involves a method for treating or preventing a vascular disease in a mammal that includes administering to the mammal a therapeutically effective amount of Tenascin-C.

Moreover, as described herein, platelet-derived growth factor (PDGF) can induce the expression of tenascin-C in cardiac endothelial cells and increases cardiac vascular patterns of tenascin-C in the endogenous heart. Hence, the compositions and methods of the invention can include effective amounts of platelet derived growth factor.

Another aspect of the invention involves, in vivo biopanning with a phage peptide library. Such biopanning was employed to identify peptides that can selectively home to bone marrow as well as cardiac vascular beds. These studies resulted in enrichment of a phage peptide sequence, designated ψR3Y32, with homology to alpha-8 integrin. Alpha-8-integrin and the ψR3Y32 peptide bind to Tenascin-C. Hence, in one embodiment, the invention provides peptides that include the amino acid sequence STISHN (SEQ ID NO:1). The peptide may be in a disulfide bond constrained configuration. According to the invention, the peptide binds to a target protein that is expressed to a larger extent in the bone marrow and cardiac tissue of younger mammals (e.g., 3-month-old mice) than in older mammals (e.g. 18-month-old mice). The target protein to which the peptides of the invention can bind and that is expressed in an age-specific manner is tenascin-C, an extracellular matrix protein that binds alpha-8 integrin.

Thus, one aspect of the invention involves peptides that home and can bind to Tenascin-C, cardiac vessels and/or bone marrow cells. Another aspect of the invention is a peptide that can bind to cells with cardioplastic potential. One example of a peptide with all these properties is the STISHN peptide with SEQ ID NO:1. According to the invention, such peptides can be used to deliver therapeutic agents to cardiac vessels and bone marrow. Such peptides can also be used to identify cells with cardioplastic portential. Moreover, in some embodiments, the peptides can modulate the activity of the target biomolecules to which they bind. For example, such peptides may inhibit or otherwise modulate the activity of Tenascin-C. As described herein, Tenascin-C promotes cell migration and delays cell adhesion. Peptides that modulate Tenascin-C activity may therefore be used for treating and preventing metastasis of cancerous cells.

### DESCRIPTION OF THE FIGURES

FIG. 1 illustrates the generalized strategy for in vivo bone marrow biopanning procedure employed for isolating peptides that can bind to bone marrow and heart tissues. A pool of phage, each displaying a different peptide, were injected into the tail vein of 3 month and 18 month old mice (1). Phage that bound to bone marrow were harvested, plated and DNA from individual plaques was sequenced (2). The clonal frequency of specific sequences for young versus old mice was noted (3). Expanded populations of phage that were differentially distributed toward binding either young or old bone marrow were separately injected into mice (4) and the titer of phage bound to bone marrow was determined (5). Phage that bound in an age-specific manner (6) were further characterized by observing their binding patterns in 18 month mice that had received 3 month old bone marrow, by searching for proteins homologous to the displayed peptide sequence and by screening bone marrow cDNA libraries with the phage or interest.

FIG. 2 graphically illustrates the binding patterns of 300 initial phage isolates in young (3 month old) and old (18 month old) mouse bone marrow. The pie chart shows to relative percentage of phage that exhibited preferential or increased (upward arrow) binding for young or old bone marrow as opposed to diminished or decreased (downward arrow) binding for young or old bone marrow. As illustrated, about 83% of the phage isolated in initial screens exhibited a low level of binding for both young and old bone marrow. About 9% of initial isolates exhibited preferential binding for both young and old bone marrow. About 5% of initial isolates exhibited higher levels of binding for young bone marrow as opposed to old bone marrow. And, about 3% of initial isolates exhibited preferential binding for old bone marrow as opposed to young bone marrow.

FIGS. 3A-D illustrate that ψR3Y32 selectively binds to tenascin-C in the heart. FIG. 3A shows that the young heart-homing phage ψR3Y32 epitope has sequence homology to extracellular region of a8-integrin. FIG. 3A also graphically illustrates that significantly more ψR3Y32 phage bind to young, rather than old, hearts as measured by titers of phage eluted from 3- and 18-month-old hearts of mice receiving tail vein injections of ψR3Y32 phage (n=4 per group). The symbol * indicates that P=0.001. FIG. 3B illustrates that labeled antibodies directed against tenascin-C (red in original) and phage coat protein pIII (green in original) stain the same regions of the young rodent heart. These data indicate that ψR3Y32 binds to areas of tenascin-C expression in cardiac venules and microvessels. The smaller inserts illustrate separate signals for tenascin-C and pII immunostains, respectively. FIG. 3C provides a close-up view of tenascin-C expression in a venule of the heart. FIG. 3D shows that tenascin-C and the nuclear marker 4,6-diamidino-2-phenylindole (DAPI) co-stain demonstrate luminal expression of tenascin-C in an arteriole. These data confirm that tenascin-C is expressed on the luminal surface of cardiac vessels, and therefore may act at sites of endothelial cell/endothelial progenitor cell incorporation during vascular remodeling. The anti-tenascin-C antibody used in for FIGS. 3B and D binds to the C-terminal region of tenascin-C whereas the anti-tenascin-C antibody used for FIG. 3C reacts with the N-terminal region of tenascin-C. The scale bars in FIGS. 3B and D were 50μm; and in FIG. 3C the scale bar was 100 μm.

FIGS. 4A-F illustrates that endothelial progenitor cells incorporate at sites of tenascin-C expression in the heart. FIG. 4A shows tenascin-C expression in venule of a young PDGF-treated mouse heart sacrificed 24 h after tail vein injection of whole lacZ-positive bone marrow from a young ROSA-26 mouse. FIG. 4B shows β-gal-expression in venule, demonstrating integration of donor bone marrow-derived endothelial cells into the cardiac vasculature. FIG. 4C illustrates that tenascin-C and β-gal expression co-localize in the sections depicted in FIGS. 4A and 5B. FIG. 4D illustrates tenascin-C expression in venule of a PDGF-treated young heart. FIG. 4E shows PDGFRα expression in same venule shown in FIG. 4D. FIG. 4F illustrates co-localization of tenascin-C and PDGFRα expression in the same sections shown in FIGS. 4D and 4E. The scale bars in FIGS. 4C and F are 50 μm.

FIGS. 5A-E illustrates that cardiac microvascular endothelial cells display diminished attachment and spreading on tenascin-C a few hours after plating, but not after about twenty-four hours. Rat cardiac endothelial cells were cultured on collagen (FIGS. 5A and B) or tenascin-C (FIGS. 5D and E) at 10 μg/ml for 3 h (FIGS. 5A and C) or 24 h (FIGS. 5B and D). While cells plated on collagen readily attached and formed a monolayer, this process was delayed in cells cultured on tenascin-C. FIG. 5E graphically illustrates that more cells fail to attach to a tenascin-C substrate (dark bars) than to a collagen substrate (light bars). Cell adhesion was analyzed after plating cardiac endothelial cells on collagen or tenascin-C (n=4-8 per group) for ten minutes or 3 hours. The symbol * indicates P<0.0001.

FIGS. 6A-L further illustrates the time period and effect of substrate concentration on attachment and spreading of cardiac microvascular endothelial cells. Rat cardiac microvascular endothelial cells were cultured on 1 μg/ml, 10 μg/ml and 100 μg/ml of collagen (FIGS. 6A-C and 7G-I) or tenascin-C (FIGS. 6D-F and 6J-L). The attachment of these cells to the collagen and tenascin-C substrates was then observed 3 h (FIGS. 6A-F) or 24 h (FIGS. 6G-L) later. As shown in FIGS. 6A-C, within 3 h, cells were well attached to collagen. However, at 3 h, cells cultured on tenascin-C remained rounded, especially at higher concentrations (FIGS. 6D-F). By 24 h, cells on lower concentrations of tenascin-C have become well-attached and largely have spread into a monolayer (FIGS. 6J-K), while those cells cultured on 100 μg/ml tenascin-C form multi-layered cell clusters.

FIG. 7 illustrates that cardiac microvascular endothelial cell attachment is likely mediated by integrin and annexin II interactions. Cardiac endothelial cells cultured on collagen (FIGS. 7A-D) or tenascin-C (FIGS. 7E-H) for 3 h and 24 h express both αv-integrin (FIGS. 7A, B, E, F) and annexin II (FIGS. 7C, D, G, H). Note the diffuse patterning of annexin II in well-spread cells (FIGS. 7C, D, H), compared to higher expression at the periphery of rounded cells cultured on tenascin-C for 3 h (FIG. 7G).

FIGS. 8A-C illustrates that cardiac microvascular endothelial cells exhibit increased migratory activity when cultured on tenascin-C. FIG. 8A schematically illustrates the procedure used for evaluating migratory activity. Cardiac endothelial cells were cultured on tenascin-C or collagen for 3 h. A collagen gel matrix was overlaid onto the cell layer and angiogenic growth factors were added to the top of the gel. FIG. 8B illustrates that after 48 hours, cell migration could be seen through the gel. The arrows point out cells that were in the plane of focus within the collagen gel and the arrowheads point out cells that are above or below the plane of focus, indicating vertical migration through gel. FIG. 8C graphically illustrates the peak migratory distance of the top 5 cells cultured in the presence of either tenascin-C (dark bars) or collagen (light bars) with the indicated growth factor (GF). As shown, cells cultured on tenascin-C migrated further than those on collagen, in response to both PDGF and VEGF (n=7 per group). *, P<0.05.

### DETAILED DESCRIPTION OF THE INVENTION

The invention provides compositions useful for restoring cardiac angiogenesis and cardiac function. In one embodiment, the compositions of the invention include Tenascin-C. In another embodiment, the invention provides peptides that bind to selected biomolecules and tissues, for example, those biomolecules and tissues within discrete regions of young, but not old, mammalian heart and bone marrow tissues. In another embodiment, the invention provides peptides that bind to cells with cardioplastic potential. For example, the invention provides a peptide that has sequence homology with alpha-8 integrin that binds to the extracellular matrix protein, tenascin-C. Such peptides can selectively bind both in vitro and in vivo to endothelial cells, endothelial precursor cells, stem cells and/or the extracellular matrix. These peptides can be used as probes to identify and help isolate endothelial and other cells with cardioplastic potential. These peptides can also be used to deliver beneficial agents to heart and bone marrow tissues. In other embodiments, the peptides can be used to characterize the changing shape and profile of the endothelium in the aging vasculature, for example, by non-invasive imaging.

### DEFINITIONS

“Hybridization” as used herein means “any process by which a strand of nucleic acid joins with a complementary strand through base pairing” (Coombs J (1994) Dictionary of Biotechnology, Stockton Press, New York N.Y.).

An “insertion” or “addition” is that change in a nucleotide or amino acid sequence that has resulted in the addition of one or more nucleotides or amino acid residues, respectively.

A “phage-display library” is a protein expression library that expresses a collection of peptide sequences as fusion proteins joined with a phage coat protein. Thus, in the context of the invention, a combinatorial library of peptide sequences is expressed on the exterior of the phage particle. Those of skill in the art will recognize that phage clones that express peptides specific for selected tissues can be substantially purified by serial rounds of phage binding to those tissues. “Polynucleotide”, “nucleotide” and “nucleic acid”, used interchangeably herein, is defined as a polymeric form of nucleotides of any length, either ribonucleotides or deoxyribonucleotides. These terms include a single-, double- or triple-stranded DNA, genomic DNA, cDNA, RNA, DNA-RNA hybrids, polymers comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Polynucleotides or nucleic acids of the invention may be in the form of RNA or in the form of DNA, which DNA includes cDNA, genomic DNA or synthetic DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “reporter molecule” is any labeling or signaling moiety known to one of skill in the art including chemicals, proteins, peptides, biotin, radionuclides, enzymes, fluorescent, chemiluminescent, contrast agents, liposomes, MRI, NMR, and ESR signaling agents, and chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,277,437; 4,275,149 and 4,366,241.

The peptides of the present invention selectively bind to target biomolecules in vivo. A peptide “selectively binds” a target biomolecule when it interacts with a binding domain of the target biomolecule with a greater affinity, or is more specific for that binding domain as compared with other binding domains of other physiological molecules. The phrase “is specific for” refers to the degree of selectivity shown by a peptide with respect to the number and types of interacting molecules with which the peptide interacts and the rates and extent of these reactions. The phrase “selectively binds” in the present context also means binding sufficient to be useful in the method of the invention. As is known in the art, useful selective binding, for instance, to a biomolecule, depends on both the binding affinity and the concentration of the peptide ligand achievable in the vicinity of the biomolecule. Thus, binding affinities lower than those found for any naturally occurring competing factors or ligands may be useful, as long as the cell or tissue to be treated can tolerate the concentration of administered peptide needed to permit binding to the target biomolecule.

“Stringency” typically occurs in a range from about Tm −5° C. (5° C. below the Tm of the probe) to about 20° C. to 25° C. below Tm. As will be understood by those of skill in the art, a stringent hybridization can be used to identify or detect identical polynucleotide sequences or to identify or detect similar or related polynucleotide sequences.

A “substitution” results from the replacement of one or more nucleotides or amino acids by different nucleotides or amino acids, respectively.

As used herein, a “target” is a biomolecule or tissue to which a peptide identified according to the invention can bind.

A “therapeutic agent” is any drug, compound, enzyme, protein, nucleic acid, toxin or other agent that one of skill in the art can use to beneficially treat a target mammalian tissue that can bind a peptide of the invention. Such therapeutic agents include, for example, platelet derived growth factor, thrombolytic agents such as streptokinase, tissue plasminogen activator, plasmin and urokinase, anti-thrombotic agents such as tissue factor protease inhibitors (TFPI), nematode-extracted anticoagulant proteins (NAPs) and the like, metalloproteinase inhibitors, anti-inflammatory agents or liposomes that contain platelet derived growth factor, thrombolytic agents such as streptokinase, tissue plasminogen activator, plasmin and urokinase, anti-thrombotic agents such as tissue factor protease inhibitors (TFPI), nematode-extracted anticoagulant proteins (NAPs) and the like, metalloproteinase inhibitors, or anti-inflammatory agents. In another embodiment, the therapeutic agent is a nucleic acid useful for gene therapy. Such a nucleic acid can be directly attached to a peptide of the invention, or it can be present in a phage particle, liposome or other vector available to one of skill in the art.

A “variant” peptide is defined as a peptide with an amino acid sequence that differs by one or more amino acids from a reference peptide or amino acid sequence. Variant peptides will have substantially the same physical, chemical and/or functional properties as the reference peptide. In general, a variant may have “conservative” changes, wherein a substituted amino acid has similar structural or chemical properties, for example, replacement of leucine with isoleucine. Similar minor variations may also include amino acid deletions or insertions, or both. In contrast to a variant peptide, a “derivative” peptide may have somewhat different physical, chemical and/or functional properties compared to the reference peptide. For example, a derivative peptide can have enhanced binding properties relative to the reference peptide. A derivative may therefore have “nonconservative” changes, e.g., replacement of a glycine with a tryptophan. Guidance in determining which and how many amino acid residues may be substituted, inserted or deleted to retain or enhance the physical, chemical and/or functional properties (e.g. binding properties) of a peptide is provided herein and is available in the art, for example, in certain computer programs such as DNAStar.

Vascular Diseases

According to the invention, tenascin-C can restore cardiac angiogenic function in a mammal that has diseased or senescent cardiac angiogenic function. As illustrated herein, tenascin-C can modulate endothelial cell migration and adhesion, promoting remodeling of vascular tissues and rejuvenating cardiac and other vascular tissues. Hence, Tenascin-C and compositions thereof can be used to treat and prevent vascular diseases.

In some embodiments, Tenascin-C is formulated into a composition that can also contain platelet derived growth factor and/or cells that have cardioplastic potential. Cells that have cardiovascular potential can be isolated using the peptides of the invention (e.g., peptides with SEQ ID NO:1) as described in more detail below. Cells with cardioplastic potential include cells that have angiogenic potential, that can help remodel vascular tissues, that help repair or develop blood vessels, or promote the repair or regeneration of cardiac muscle. Examples of cells with cardioplastic potential include endothelial progenitor cells, stem cells, bone marrow cells, hematopoietic stem cells, embryonic stem cell lines, cardiac microvascular endothelial cells and the like. Hence, compositions using Tenascin-C and cells with cardioplastic potential can be used to treat and prevent vascular diseases.

The vascular diseases treated by the present invention are vascular diseases of mammals. The word mammal means any mammal. Some examples of mammals include, for example, pet animals, such as dogs and cats; farm animals, such as pigs, cattle, sheep, and goats; laboratory animals, such as mice and rats; primates, such as monkeys, apes, and chimpanzees; and humans. In some embodiments, humans are preferably treated with the compositions and methods of the invention.

According to the invention, endothelial cells within normal vascular tissues change as they grow older, exhibiting reduced angiogenesis, reduced capacity for re-endothelization and losing their ability to communicate with other cells by secreting signaling agents. These changes can lead to a diminished capacity for blood vessel formation, a reduction in blood flow to the associated organ or system, and an inability to recover from injuries or diseases that adversely affect blood vessels.

Accordingly, the invention relates to methods for treating a vascular condition, or a circulatory condition, such as a condition associated with loss, injury or disruption of the vasculature within an anatomical site or system. The term “vascular condition” or “vascular disease” refers to a state of vascular tissue where blood flow is, or can become, impaired.

Many pathological conditions can lead to vascular diseases that are associated with alterations in the normal vascular condition of the affected tissues and/or systems. Examples of vascular conditions or vascular diseases to which the compositions and methods of the invention apply are those in which the vasculature of the affected tissue or system is senescent or otherwise altered in some way such that blood flow to the tissue or system is reduced or in danger of being reduced. Examples of vascular conditions that can be treated with the compositions and methods of the invention include atherosclerosis, preeclampsia, peripheral vascular disease, erectile dysfunction, cancers, renal failure, heart disease, and stroke. Vascular, circulatory or hypoxic conditions to which the methods of the invention apply also include those associated with, but not limited to, maternal hypoxia (e.g., placental hypoxia, preeclampsia), abnormal pregnancy, peripheral vascular disease (e.g., arteriosclerosis), transplant accelerated arteriosclerosis, deep vein thrombosis, erectile dysfunction, cancers, renal failure, stroke, heart disease, sleep apnea, hypoxia during sleep, female sexual dysfunction, fetal hypoxia, smoking, anemia, hypovolemia, vascular or circulatory conditions which increase risk of metastasis or tumor progression, hemorrhage, hypertension, diabetes, vasculopathologies, surgery (e.g., per-surgical hypoxia, post-operative hypoxia), Raynaud's disease, endothelial dysfunction, regional perfusion deficits (e.g., limb, gut, renal ischemia), myocardial infarction, stroke, thrombosis, frost bite, decubitus ulcers, asphyxiation, poisoning (e.g., carbon monoxide, heavy metal), altitude sickness, pulmonary hypertension, sudden infant death syndrome (SIDS), asthma, chronic obstructive pulmonary disease (COPD), congenital circulatory abnormalities (e.g., Tetralogy of Fallot) and Erythroblastosis (blue baby syndrome). In particular embodiments, the invention is directed to compositions and methods of treating loss of circulation or endothelial dysfunction in an individual.

Thus, the invention is directed to compositions and methods of treating diseases such as stroke, atherosclerosis, acute coronary syndromes including unstable angina, thrombosis and myocardial infarction, plaque rupture, both primary and secondary (in-stent) restenosis in coronary or peripheral arteries, transplantation-induced sclerosis, peripheral limb disease, intermittent claudication and diabetic complications (including ischemic heart disease, peripheral artery disease, congestive heart failure, retinopathy, neuropathy and nephropathy), or thrombosis.

In some embodiments, the vascular condition or vascular disease arises from damaged myocardium. As used herein “damaged myocardium” refers to myocardial cells that have been exposed to ischemic conditions. These ischemic conditions may be caused by a myocardial infarction, or other cardiovascular disease. The lack of oxygen causes the death of the cells in the surrounding area, leaving an infarct that can eventually scar.

Preferably, damaged myocardium is treated with the methods and compositions of the invention before damage occurs (e.g. when damage is suspected of occurring) or as quickly as possible after damage occurs. Hence, the methods and compositions of the invention are advantageously employed on aged heart tissues that are in danger of ischemia, heart attack or loss of blood flow. The methods and compositions of the invention are also advantageously employed on recently damaged myocardium and on not so recently damaged myocardium.

As used herein “recently damaged myocardium” refers to myocardium that has been damaged within one week of treatment being started. In a preferred embodiment, the myocardium has been damaged within three days of the start of treatment. In a further preferred embodiment, the myocardium has been damaged within twelve hours of the start of treatment.

The methods and compositions of the invention can be used to prevent or to treat these vascular conditions. These methods involve administering an effective amount of Tenascin-C, cells with cardioplastic potential or cells with cardioplastic potential that are treated with Tenascin-C. Such cells with cardioplastic potential include cells that have angiogenic potential, that can help remodel vascular tissues, that help repair or develop blood vessels, or that promote the repair or regenerration of cardiac muscle. Examples of cells with cardioplastic potential include endothelial progenitor cells, stem cells, bone marrow cells, hematopoietic stem cells, embryonic stem cell lines, cardiac microvascular endothelial cells and the like. Such compositions of Tenascrin-C and/or cells can be administered alone or in combination with platelet-derived growth factor (PDGF). An effective amount of Tenascin-C, cells with cardioplastic potential or cells with cardioplastic potential that are treated with Tenascin-C is an amount that effectively stimulates the migration of endothelial cells (including endothelial progenitor cells) or restores some vascularization in a tissue.

Tenascin-C

In some embodiments, the invention provides compositions containing tenascin-C and methods of using such compositions for treating cardiac and/or vascular conditions. Tenascin-C is expressed both during embryonic development and in the adult at sites of dynamic cell migration and remodeling. For a review, see Jones, F. S. and P. L. Jones, The tenascin family of ECM glycoproteins: structure, function, and regulation during embryonic development and tissue remodeling. Dev Dyn, 2000. 218(2): p. 235-59. Tenascin-C is expressed post-natally at sites of wound healing in tissues such as skin, bone, muscle. Tenascin-C is also expressed in the heart by myofibroblasts within the first twenty four hours after myocardial infarction. Chiquet-Ehrismann, R. and M. Chiquet, Tenascins: regulation and putative functions during pathological stress. J Pathol, 2003. 200(4): p. 488-99; Imanaka-Yoshida, K., et al., Tenascin-C modulates adhesion of cardiomyocytes to extracellular matrix during tissue remodeling after myocardial infarction. Lab Invest, 2001. 81(7): p. 1015-24. Moreover, tenascin-C knockout mice display defects in wound healing and exhibit impaired hematopoiesis in vitro. Mackie, E. J., W. Halfter, and D. Liverani, Induction of tenascin in healing wounds. J Cell Biol, 1988. 107(6 Pt 2): p. 2757-67; Ohta, M., et al., Suppression of hematopoietic activity in tenascin-C-deficient mice. Blood, 1998. 91(11): p. 4074-83. In the bone marrow, tenascin-C is expressed in the stroma and is adhesive to hematopoietic cells. Klein, G., S. Beck, and C. A. Muller, Tenascin is a cytoadhesive extracellular matrix component of the human hematopoietic microenvironment. J Cell Biol, 1993. 123(4): p. 1027-35.

Hematopoietic progenitor cells and endothelial progenitor cells utilize common mechanisms of mobilization and may also derive from a common progenitor. Eichmann, A., et al., Ligand-dependent development of the endothelial and hemopoietic lineages from embryonic mesodermal cells expressing vascular endothelial growth factor receptor 2. Proc Natl Acad Sci U S A, 1997. 94(10): p. 5141-46; Choi, K., et al., A common precursor for hematopoietic and endothelial cells. Development, 1998. 125(4): p. 725-32. Impaired mobilization of hematopoietic stem cells in mice with null mutations for tenascin-C may be concomitant with a loss of endothelial progenitor cell generation and/or mobilization, which, according to the invention, accounts for defective wound healing and angiogenesis in these mice. Moreover, tenascin-C is expressed in a wide range of tumors at the leading edge of angiogenesis. Together, these findings suggest that tenascin-C may play important roles both in local repair of the heart after myocardial infarction as well as generation and mobilization of endothelial progenitor cells from the bone marrow to promote angiogenesis.

While some embodiments of the invention are directed to use of Tenascin-C, other binding partners for α8-integrin, such as vitronectin, can also be involved in the pathways described above and play a role in cardiac angiogenesis. Hence, the invention contemplates using vitronectin in compositions and for treating cardiac and vascular diseases.

Tenascin-C is a 1.1 to 1.5 million dalton, hexameric glycoprotein that is located primarily in the extracellular matrix. Human, mouse, and chicken tenascin-C contain a cysteine-rich segment at their amino termini through which six tenascin-C monomers link into a hexamer. This segment is followed by epidermal growth factor-like repeats, fibronectin-type III repeats (FN-L) and a globular carboxyl terminus, homologous to fibrinogen (Fbg-L). Erickson et al. (1989) Annu. Rev. Cell Biol. 5, 71-92. These domains mediate the interaction between the tenascin-C molecule and cells. For example, endothelial cells interact with tenascin-C through its fibrinogen-like domain. Joshi et al. (1993) J. Cell Sci., 106(Pt 1), 389-400. In contrast, the FN-L domain of tenascin-C mediates interaction with fibroblasts. Aukhil et al. (1993) J. Biol. Chem. 268(4), 2542-53.

Tenascin-C is expressed during embryogenesis, wound healing, and neoplasia, suggesting a role for this protein in tissue remodeling (Erickson & Bourdon, (1989) Ann Rev Cell Biol 5:71-92). Neoplastic processes also involve tissue remodeling, and tenascin-C is over-expressed in many tumor types including carcinomas of the lung, breast, prostate, and colon, astrocytomas, glioblastomas, melanomas, and sarcomas (Soini et al., (1993) Am J Clin Pathol 100(2):145-50; Koukoulis et al., (1991) Hum Pathol 22(7):636-43; Borsi et al., (1992) Int J Cancer 52(5):688-92; Koukoulis et al., (1993) J Submicrosc Cytol Pathol 25(2):285-95; Ibrahim et al., (1993) Hum Pathol 24(9):982-9; Riedl et al., (1998) Dis Colon Rectum 41(1):86-92; Tuominen & Kallioinen (1994) J Cutan Pathol 21(5):424-9; Natali et al., (1990) Int J Cancer 46(4):586-90; Zagzag et al., (1995) Cancer Res 55(4):907-14; Hasegawa et al., (1997) Acta Neuropathol (Berl) 93(5):431-7; Saxon et al., (1997) Pediatr Pathol Lab Med 17(2):259-66; Hasegawa et al., (1995) Hum Pathol 26(8):838-45).

In addition, tenascin-C is overexpressed in hyperproliferative skin diseases, e.g. psoriasis (Schalkwijk et al., (1991) Br J Dermatol 124(1):13-20), and in atherosclerotic lesions (Fukumoto et al., (1998) J Atheroscler Thromb 5(1):29-35; Wallner et al., (1999) Circulation 99(10):1284-9). Radiolabeled antibodies that bind tenascin-C are used for imaging and therapy of tumors in clinical settings (Paganelli et al., (1999) Eur J Nucl Med 26(4):348-57; Paganelli et al., (1994) Eur J Nucl Med 21(4):314-21; Bigner et al., (1998) J Clin Oncol 16(6):2202-12; Merlo et al., (1997) Int J Cancer 71(5):810-6).

According to the invention, tenascin-C is expressed in an age-dependent manner in bone marrow cells and in cardiac microvascular tissues. Also according to the invention, tenascin-C can maintain cardiac endothelial cells, and possibly bone-marrow derived endothelial precursor cells (EPCs), in an undifferentiated state. Cells maintained in such an undifferentiated state have diminished cell adhesion and are available as precursor cells that are useful in vascular remodeling. Further according to the invention, Tenascin-C may promote cardiac angiogenesis via local and/or systemic mechanisms.

The invention therefore provides a method of restoring cardiac angiogenic function in a patient having senescent cardiac angiogenic function. The method involves administering to the patient a therapeutically effective amount of tenascin-C.

The tenascin-C employed can be any mammalian tenascin-C polypeptide including, for example, human, mouse, rat, rabbit, goat, bovine, horse, sheep and any other mammalian tenascin-C polypeptide. One example of a sequence for human tenascin-C can be found in the NCBI database at accession number NP 002151, gi:4504549. See website at www.ncbi.nih.nlm.gov. This amino acid sequence of a human tenascin-C (SEQ ID NO:2) is provided below:

| | | | | |
|---|---|---|---|---|
| 1 | MGAMTQLLAG | VFLAFLALAT | EGGVLKKVIR | HKRQSGVNAT |
| 41 | LPEENQPVVF | NHVYNIKLPV | GSQCSVDLES | ASGEKDLAPP |
| 81 | SEPSESFQEH | TVDGENQIVF | THRINIPRRA | CGCAAAPDVK |
| 121 | ELLSRLEELE | NLVSSLREQC | TAGAGCCLQP | ATGRLDTRPF |
| 161 | CSGRGNFSTE | GCGCVCEPGW | KGPNCSEPEC | PGNCHLRGRC |
| 201 | IDGQCICDDG | FTGEDCSQLA | CPSDCNDQGK | CVNGVCICFE |
| 241 | GYAGADCSRE | ICPVPCSEEH | GTCVDGLCVC | HDGFAGDDCN |
| 281 | KPLCLNNCYN | RGRCVENECV | CDEGFTGEDC | SELICPNDCF |
| 321 | DRGRCINGTC | YCEEGFTGED | CGKPTCPHAC | HTQGRCEEGQ |
| 361 | CVCDEGFAGL | DCSEKRCPAD | CHNRGRCVDG | RCECDDGFTG |
| 401 | ADCGELKCPN | GCSGHGRCVN | GQCVCDEGYT | GEDCSQLRCP |
| 441 | NDCHSRGRCV | EGKCVCEQGF | KGYDCSDMSC | PNDCHQHGRC |
| 481 | VNGMCVCDDG | YTGEDCRDRQ | CPRDCSNRGL | CVDGQCVCED |
| 521 | GFTGPDCAEL | SCPNDCNGQG | RCVNGQCVCH | EGFMGKDCKE |
| 561 | QRCPSDCHGQ | GRCVDGQCIC | HEGFTGLDCG | QHSCPSDCNN |
| 601 | LGQCVSGRCI | CNEGYSGEDC | SEVSPPKDLV | VTEVTEETVN |
| 641 | LAWDNEMRVT | EYLVVYTPTH | EGGLEMQFRV | PGDQTSTIIQ |
| 681 | ELEPGVEYFI | RVFAILENKK | SIPVSARVAT | YLPAPEGLKF |
| 721 | KSIKETSVEV | EWDPLDIAFE | TWEIIFRNMN | KEDEGEITKS |
| 761 | LRRPETSYRQ | TGLAPGQEYE | ISLHIVKNNT | RGPGLKRVTT |
| 801 | TRLDAPSQIE | VKDVTDTTAL | ITWFKPLAEI | DGIELTYGIK |
| 841 | DVPGDRTTID | LTEDENQYSI | GNLKPDTEYE | VSLISRRGDM |
| 881 | SSNPAKETFT | TGLDAPRNLR | RVSQTDNSIT | LEWRNGKAAI |
| 921 | DSYRIKYAPI | SGGDHAEVDV | PKSQQATTKT | TLTGLRPGTE |
| 961 | YGIGVSAVKE | DKESNPATIN | AATELDTPKD | LQVSETAETS |
| 1001 | LTLLWKTPLA | KFDRYRLNYS | LPTGQWVGVQ | LPRNTTSYVL |
| 1041 | RGLEPGQEYN | VLLTAEKGRH | KSKPARVKAS | TEQAPELENL |
| 1081 | TVTEVGWDGL | RLNWTAADQA | YEHFIIQVQE | ANKVEAARNL |
| 1141 | TVPGSLRAVD | IPGLKAATPY | TVSIYGVIQG | YRTPVLSAEA |
| 1181 | STGETPNLGE | VVVAEVGWDA | LKLNWTAPEG | AYEYFFIQVQ |
| 1201 | EADTVEAAQN | LTVPGGLRST | DLPGLKAATH | YTITIRGVTQ |
| 1241 | DFSTTPLSVE | VLTEEVPDMG | NLTVTEVSWD | ALRLNWTTPD |
| 1281 | GTYDQFTIQV | QEADQVEEAH | NLTVPGSLRS | MEIPGLRAGT |
| 1321 | PYTVTLHGEV | RGHSTRPLAV | EVVTEDLPQL | GDLAVSEVGW |
| 1361 | DGLRLNWTAA | DNAYEHFVIQ | VQEVNKVEAA | QNLTLPGSLR |
| 1401 | AVDIPGLEAA | TPYRVSIYGV | IRGYRTPVLS | AEASTAKEPE |
| 1441 | IGNLNVSDIT | PESFNLSWMA | TDGIFETFTI | EIIDSNRLLE |
| 1481 | TVEYNISGAE | RTAHISGLPP | STDFIVYLSG | LAPSIRTKTI |
| 1521 | SATATTEALP | LLENLTISDI | NPYGFTVSWM | ASENAFDSFL |
| 1561 | VTVVDSGKLL | DPQEFTLSGT | QRKLELRGLI | TGIGYEVMVS |
| 1601 | GFTQGHQTKP | LRAEIVTEAE | PEVDNLLVSD | ATPDGFRLSW |
| 1641 | TADEGVFDNF | VLKIRDTKKQ | SEPLEITLLA | PERTRDLTGL |
| 1681 | REATEYEIEL | YGISKGRRSQ | TVSAIATTAM | GSPKEVIFSD |
| 1721 | ITENSATVSW | RAPTAQVESF | RITYVPITGG | TPSMVTVDGT |
| 1761 | KTQTRLVKLI | PGVEYLVSII | AMKGFEESEP | VSGSFTTALD |
| 1801 | GPSGLVTANI | TDSEALARWQ | PAIATVDSYV | ISYTGEKVPE |
| 1841 | ITRTVSGNTV | EYALTDLEPA | TEYTLRIFAE | KGPQKSSTIT |
| 1881 | AKFTTDLDSP | RDLTATEVQS | ETALLTWRPP | RASVTGYLLV |
| 1921 | YESVDGTVKE | VIVGPDTTSY | SLADLSPSTH | YTAKIQALNG |
| 1961 | PLRSNMIQTI | FTTIGLLYPF | PKDCSQAMLN | GDTTSGLYTI |
| 2001 | YLNGDKAQAL | EVFCDMTSDG | GGWIVFLRRK | NGRENFYQNW |
| 2041 | KAYAAGFGDR | REEFWLGLDN | LNKITAQGQY | ELRVDLRDHG |
| 2081 | ETAFAVYDKF | SVGDAKTRYK | LKVEGYSGTA | GDSMAYHNGR |
| 2121 | SFSTFDKDTD | SAITNCALSY | KGAFWYRNCH | RVNLMGRYGD |
| 2161 | NNHSQGVNWF | HWKGHEHSIQ | FAEMKLRPSN | FRNLEGRRKR |
| 2201 | A | | | |

A nucleic acid that encodes a human tenascin-C can be found in the NCBI database at accession number NM 002160, gi:4504548. See website at www.ncbi.nih.nlm.gov.

Immunohistochemical data provided herein shows that tenascin-C is expressed in the normal adult heart, particularly within the cardiac vasculature. Interestingly, expression is located on the luminal surface of the vessels, as would be expected for a factor able to bind circulating phage. There has been much investigation of the specific actions of tenascin-C on a variety of cell types, often revealing seemingly contradictory effects. Tenascin-C promotes attachment of human umbilical endothelial cells (HUVECs) and fibroblasts in vitro, for example (Sriramarao et al., 1993; Bourdon and Ruoslahti, 1989), but appears to be anti-adhesive for oligodendrocytes and neural crest cells (Kiernan et al., 1996; Tan et al., 1987; Erickson and Bourdon, 1989). Similarly, tenascin-C promotes migration of HUVECs and bovine retinal endothelial cells (Sriramarao et al., 1993; Castellon et al., 2002) but inhibits migration of glioma cells and oligodendrocytes (Zagzag et al., 2002; Joshi et al., 1993). In vivo, tenascin-C is often found as a component of the migratory environment of motile cells, such as hematopoietic progenitor cells and neuronal cells in the developing embryo, but can also represent a restrictive zone through which cells do not migrate, such as the anterior half of the somite (Tan et al., 1997). The diversity of actions of tenascin-C according to cell type and environment is likely due to a number of factors, including the binding partners available, variation in the isoforms of tenascin-C expressed and enzymatic processing of these isoforms by various proteinases, including the MMPs, to either expose or destroy various domains regulating cell activity (Imai et al., 1994; Siri et al., 1995).

The present invention represents the first elucidation of the specific role of tenascin-C in regulating the phenotype of cardiac vascular endothelial cells. Data provided herein shows that tenascin-C is anti-adhesive for these cells at early time-points, but that this effect is lost within 24 hours, by which time cells readily adhere and spread on tenascin-C. The interaction of annexin II with the alternatively spiced region of tenascin-C is known to inhibit cell adhesion (Chung et al., 1994, 1996). This is consistent with a change in annexin II expression for cells plated on tenascin-C for three hours, when the vast majority of cells are still rounded, and for twenty-four hours, when most cells exhibit a flattened, well-attached phenotype. This switch in morphology may be due to proteolytic processing of tenascin-C by one or a combination of proteases. Tenascin-C itself is known to upregulate MMP expression (Tremble et al., 1994; Kalembeyi et al., 2003). Additionally, annexin II has been shown to form a complex with the cysteine protease cathepsin B, which cleaves various extracellular matrix proteins, including tenascin-C (Mai et al., 2000, 2002). Thus, either upregulation of MMP expression and/or interaction with cathepsin during this period, and subsequent proteolysis of tenascin-C, may disrupt annexin II binding domains or expose pro-adhesive domains, thereby inducing a change in endothelial cell morphology.

The elucidation of the ability of tenascin-C to regulate cardiac endothelial cell phenotype has provided insight into the role of cardiac tenascin-C in vivo. The data provided herein demonstrate the presence of tenascin-C at sites of endothelial progenitor cell incorporation into the cardiac vasculature, suggesting a potential role for this molecule in cell attachment and invasion. Moreover, the co-localization of tenascin-C with PDGFRα-positive endothelial cells and upregulation of cardiac vascular tenascin-C expression by PDGF treatment suggests that tenascin-C may be an important downstream component of PDGF-mediated cardioprotective mechanisms. The inventors have previously shown that these mechanisms are compromised in the aging heart, but can be restored by upregulation of PDGF signaling. This results in increased angiogenesis and limits the extent of myocardial infarction (Edelberg et al., 2002). Upregulation of PDGF expression in response to vascular injury may increase tenascin-C expression and thereby provides a platform for homing endothelial progenitor cells and remodeling endothelial cells as they arrive at the site of damage. Here, the early modulation of endothelial cell morphology to promote a rounded cell shape with limited adhesion in turn promotes cell migration. These data indicate tenascin-C may play a role in the angiogenic switch, facilitating incorporation of endothelial progenitor cells or local endothelial cells into the remodeling vasculature and promoting their migratory ability.

Thus, tenascin-C likely has an important role in cardiac vascular repair mechanisms. Antibodies against tenascin-C and peptides that bind tenascin-C may inhibit tumor angiogenesis in patients with gliomas. Enhancement of tenascin-C expression in a site-specific manner may have therapeutic benefits in the treatment of cardiovascular disease.

Peptides

Peptides were isolated by in vivo methods that bound to selected biomolecules and tissues of interest. In many instances, such peptides selectively bind to such biomolecules and tissues. In some embodiments, a peptide that selectively binds to a biomolecule or tissue of interest, binds with sufficient selectivity to permit the peptide to become localized in vivo at the site of the biomolecule or tissue. Peptides that selectively bind to biomolecules and tissues can therefore be detected at the site of such biomolecules and tissues. Desirable peptides that selectively bind to biomolecules and tissues permit reliable detection of those biomolecules and tissues in vivo. Desirable peptides that selectively bind to biomolecules and tissues may also permit reliable delivery of a therapeutic agent to the site of the biomolecule or tissue. However, because various types of reporter molecules and therapeutic agents may alter the physical and chemical properties of the peptide or sterically hinder binding, a peptide conjugated to such a reporter molecule or therapeutic agent may still “selectively bind” even though some modification of the peptide conjugate, reporter molecule or therapeutic agent is needed to optimize binding.

An in vivo phage biopanning study was performed as described herein. The aim of the study was to identify peptides that may play important roles in regulating endothelial cell and/or endothelial progenitor cell phenotype and subsequent cardiac angiogenic mechanisms. Peptides of interest were expressed at important sites of endothelial cell phenotype modulation, for example, areas in which endothelial cells or progenitor cells exhibit dynamic changes in morphology and/or behavior to promote systemic or local angiogenic and/or vasculogenic mechanisms. Peptides of interest bound specifically on the surface of the vasculature to extracellular domains that are involved in modulating endothelial cell attachment, migration and function at the time of cellular incorporation into remodeling vasculature. Finally, peptides that bound preferentially to cardiac vasculature in young host animals were sought, because these peptides were more likely to represent pro-angiogenic factors.

Thus, a phage display library was introduced into mice and after circulation of the library, phage were eluted from heart tissues of the mice, where they bind to the cardiac vasculature, as well as from the bone marrow, where endothelial progenitor cells are generated in response to vascular injury. Shi, Q., et al., Evidence for circulating bone marrow-derived endothelial cells. Blood, 1998. 92(2): p. 362-67; Gunsilius, E., et al., Evidence from a leukaemia modelfor maintenance of vascular endothelium by bone-marrow-derived endothelial cells. Lancet, 2000. 355(9216): p. 1688-91; Shintani, S., et al., Augmentation of postnatal neovascularization with autologous bone marrow transplantation. Circulation, 2001. 103(6): p. 897-903. To ensure that clones isolated from the eluted phage would specifically bind to significant biomolecules on the surface of cardiac vasculature, isolates were eluted four minutes after injection to allow full circulation of the phage through the host vasculature but avoid phage traversal of the endothelium of the vessels.

Examples of peptides that can selectively bind to biomolecules and tissues within discrete regions of young, but not old, mammalian heart and bone marrow tissues include peptides having sequence homology with a region of alpha-8 integrin that binds to the extracellular matrix protein, tenascin-C. For example, such a peptide was obtained from phage ψR3Y32 by the biopanning strategy described above. The ψR3Y32 phage preferentially bound young cardiac and bone marrow vasculature and displayed a peptide with the amino acid sequence STISHN (SEQ ID NO:1). The SEQ ID NO:1 peptide shared homology with a region of alpha-8 integrin and cab bind to tenascin-C. Peptides isolated as described herein can selectively bind both in vitro and in vivo to Tenascin-C, endothelial cells and/or the extracellular matrix.

The invention is also directed to variants and derivatives of the isolated peptides that can bind to the biomolecule or tissue to which the isolated peptide bound. Such variants and derivatives have identity with at least about four of the amino acid positions of SEQ ID NO:1 and are capable of binding to young, but not old, mammalian heart and bone marrow tissues. In a preferred embodiment, the variants and derivatives have identity with at least about five of the amino acid positions of SEQ ID NO:1 and can bind to young, but not old, mammalian heart and bone marrow tissues.

Amino acid residues of the isolated peptides and variants or derivatives thereof can be genetically encoded L-amino acids, naturally occurring non-genetically encoded L-amino acids, synthetic L-amino acids or D-enantiomers of any of the above. The amino acid notations used herein for the twenty genetically encoded L-amino acids and common non-encoded amino acids are conventional and are as shown in Table 1.

TABLE 1

| Amino Acid | One-Letter Symbol | Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| 2,3-Diaminopropionic acid | | Dpr |
| α-Aminoisobutyric acid | | Aib |
| N-Methylglycine (sarcosine) | | MeGly |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydroisoquinoline-3- | | Tic |
| carboxylic acid | | |
| β-2-thienylalanine | | Thi |
| Methionine sulfoxide | | MSO |
| Homoarginine | | hArg |
| N-acetyl lysine | | AcLys |
| 2,4-Diamino butyric acid | | Dbu |
| P-Aminophenylalanine | | Phe(pNH₂) |
| N-methylvaline | | MeVal |
| Homocysteine | | hCys |
| Homoserine | | hSer |
| E-Amino hexanoic acid | | Aha |
| δ-Amino valeric acid | | Ava |
| 2,3-Diaminobutyric acid | | Dab |

Peptides that are encompassed within the scope of the invention can have one or more amino acids substituted with an amino acid of similar or different chemical and/or physical properties, so long as these variant and derivative peptides retain the ability to bind to young, but not old, mammalian heart and bone marrow tissues.

When generating a variant or derivative peptide, amino acids that reside within similar classes or subclasses can be substituted for amino acids in a reference peptide or amino acid sequence. As known to one of skill in the art, amino acids can be placed into three main classes: hydrophilic amino acids, hydrophobic amino acids and cysteine-like amino acids, depending primarily on the characteristics of the amino acid side chain. These main classes may be further divided into subclasses.

Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated π-electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include glycine, proline and methionine. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but where a bond in the side chain has a pair of electrons that are held more closely by one of the atoms involved in the bond. Examples of genetically encoded polar amino acids include asparagine and glutamine. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. An example of a genetically encoded cysteine-like amino acid is cysteine. Examples of non-genetically encoded cysteine-like amino acids include homocysteine and penicillamine.

As will be appreciated by those having skill in the art, the above classifications are not absolute. Several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has an apolar character. Thus, while not strictly classified as a hydrophobic or an apolar amino acid, in many instances cysteine can be used to confer hydrophobicity to a peptide.

Certain commonly encountered amino acids that are not genetically encoded and that can be present, or substituted for an amino acid, in the peptides, peptide variants and peptide derivatives of the invention include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ∈-aminohexanoic acid (Aha); δ-aminovaleric acid (Ava); N-methylglycine (MeGly); omithine (Om); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall into the categories defined above.

The classifications of the above-described genetically encoded and non-encoded amino acids are summarized in Table 2, below. It is to be understood that Table 2 is for illustrative purposes only and does not purport to be an exhaustive list of amino acid residues that may comprise the peptides, variants and derivatives described herein. Other amino acid residues that are useful for making the peptides, peptide variants and peptide derivatives described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 2

| Classifi- | Genetically | |
|---|---|---|
| cation | Encoded | Genetically Non-Encoded |
| Hydrophobic | | |
| Aromatic | F, Y, W | Phg, Nal, Thi, Tic, Phe(4-Cl), |
| | | Phe(2-F), Phe(3-F), Phe(4-F), |
| | | Pyridyl Ala, Benzothienyl Ala |
| Apolar | M, G, P | Cha |
| Aliphatic | A, V, L, I | t-BuA, t-BuG, MeIle, Nle, MeVal, |
| | | Cha, bAla, MeGly, Aib |
| Hydrophilic | | |
| Acidic | D, E | |
| Basic | H, K, R | Dpr, Orn, hArg, Phe(p-NH₂), DBU, |
| | | A₂ BU |
| Polar | Q, N, S, T, Y | Cit, AcLys, MSO, hSer |
| Cysteine- | C | Pen, hCys, β-methyl-Cys |
| Like | | |

Peptides of the invention can have any amino acid substituted by any similarly classified amino acid to create a variant or derivative peptide, so long as the peptide variant or derivative retains an ability to bind to the biomolecule or tissue to which the unaltered or reference peptide bound.
Platelet Derived Growth Factor

According to the invention, Tenascin-C compositions can include platelet derived growth factor. Moreover, cells identified and/or isolated by the methods of the invention can be treated not only with Tenascin-C but also with platelet derived growth factor prior to introduction into a patient or other mammal. Such treatment can promote differentiation of stem cells and endothelial precursor cells into cardiac myocytes that are useful for treating cardiovascular diseases.

Naturally occurring, platelet-derived growth factor is a disulfide-bonded dimer having two polypeptide chains, namely the “A” and “B” chains as well as more recently discovered C and D isoforms. The A chain is approximately 60% homologous to the B chain. Naturally occurring PDGF is found in three dimeric forms, namely PDGF-AB heterodimer, PDGF-BB homodimer, or PDGF-AA homodimer. Hannink et al., Mol. Cell. Biol., 6, 1304-1314 (1986). PDGF-AB has been identified as a predominate naturally occurring form. However, some data indicates that the PDGF-BB homodimer may be effective for wound healing. Each monomeric subunit of the biologically active dimer, irrespective of whether it is an A chain monomer or a B chain monomer, contains eight cysteine residues. Some of these cysteine residues form interchain disulfide bonds that hold the dimer together. As used herein, the term PDGF means any PDGF polypeptide or protein, including PDGF A, PDGF B, PDGF C, PDGF D, PDGF AB, PDGF BB, and PDGF AA.

The A polypeptide of human PDGF can be any mammalian PDGF A polypeptide including, for example, human, mouse, rat, rabbit, goat, bovine, horse, sheep and any other mammalian PDGF A polypeptide. The following sequence is one example of an amino acid sequence of a human PDGF A polypeptide (SEQ ID NO:3):

| | | | | |
|---|---|---|---|---|
| 1 | MRTWACLLLL | GCGYLAHALA | EEAEIPRELI | ERLARSQIHS |
| 41 | IRDLQRLLEI | DSVGAEDALE | TNLRAHGSHT | VKHVPEKRPV |
| 81 | PIRRKRSIEE | AIPAVCKTRT | VIYEIPRSQV | DPTSANFLIW |
| 121 | PPCVEVKRCT | GCCNTSSVKC | QPSRVHHRSV | KVAKVEYVRK |
| 161 | KPKLKEVQVR | LEEHLECACA | TSNLNPDHRE | EETGRRRESG |
| 201 | KKRK | | | |

A nucleic acid that encodes a human PDGF A polypeptide can be found in the NCBI database at accession number X03795, gi:35365. See website at www.ncbi.nih.nlm.gov.

The PDGF B polypeptide found in human platelets has been identified as a 109 amino acid cleavage product (PDGF-Blog) of a 241 amino acid precursor polypeptide Johnsson et al., EMBO Journal, 3(5), 921-928 (1984). An example of a human sequence for the PDGF B polypeptide is provided below (SEQ ID NO:4).

| | | | | |
|---|---|---|---|---|
| 1 | MNRCWALFLS | LCCYLRLVSA | EGDPIPEELY | EMLSDHSIRS |
| 41 | FDDLQRLLHG | DPGEEDGAEL | DLNMTRSHSG | GELESLARGR |
| 82 | RSLGSLTIAE | PAMIAECKTR | TEVFEISRRL | IDRTNANFLV |
| 121 | WPPCVEVQRC | SGCCNNRNVQ | CRPTQVQLRP | VQVRKIEIVR |
| 161 | KKPIFKKATV | TLEDHLACKC | ETVAAARPVT | RSPGGSQEQR |
| 201 | AKTPQTRVTI | RTVRVRRPPK | GKHRKFKHTH | DKTALKETLG |
| 241 | A | | | |

A nucleic acid that encodes a human PDGF B polypeptide can be found in the NCBI database at accession number X02811, gi:35371. See website at www.ncbi.nih.nlm.gov.

As recognized by one of skill in the art, these PDGF polypeptides from different mammalian species have similar amino acid sequences. According to the invention any PDGF polypeptide from any mammalian species can be utilized in the practice of the invention so long as the PDGF polypeptide can stimulate endothelial cells to promote angiogenesis.

A 109 amino acid PDGF B polypeptide is believed to be the mature form of PDGF in humans and constitutes a cleavage product of the PDGF-B precursor protein. Homology with the precursor protein begins at amino acid 82 of the 241 amino acid precursor protein and continues for 109 amino acids yielding, for example, a polypeptide with the following sequence (SEQ ID NO:5):

| | | | | |
|---|---|---|---|---|
| 82 | RSLGSLTIAE | PAMIAECKTR | TEVFEISRRL | IDRTNANFLV |
| 121 | WPPCVEVQRC | SGCCNNRNVQ | CRPTQVQLRP | VQVRKIEIVR |
| 161 | KKPIFKKATV | TLEDHLACKC | ETVAAARPVT | RSPGGSQEQR |
| 201 | AKTPQTRVTI | RTVRVRRPPK | GKHRKFKHTH | DKTALKETLG |
| 241 | A | | | |

Another form of PDGF-B (PDGF-B₁₁₉), corresponds to the first 119 amino acids of the PDGF-B precursor protein (SEQ ID NO:6):

| | | | | |
|---|---|---|---|---|
| 1 | MNRCWALFLS | LCCYLRLVSA | EGDPIPEELY | EMLSDHSIRS |
| 41 | FDDLQRLLHG | DPGEEDGAEL | DLNMTRSHSG | GELESLARGR |
| 82 | RSLGSLTIAE | PAMIAECKTR | TEVFEISRRL | IDRTNANFL |

This PDGF-B₁₁₉ form has also been identified as a major cleavage product of the precursor protein when the entire gene is encoded into a transfected mammalian host. See U.S Pat. No. 5,149,792.

Human platelet-derived growth factor is believed to be the major mitogenic growth factor in serum for connective tissue cells. PDGF can positively affect mitogenesis in arterial smooth muscle cells, fibroblast cells lines, and glial cells. Deuel et al., J. Biol. Chem., 256(17), 8896-8899 (1981). See also, e.g., Heldin et al., J. Cell Physiol., 105, 235 (1980) (brain glial cells); Raines and Ross, J. Biol. Chem., 257, 5154 (1982) (monkey arterial smooth muscle cells).

Other members of the PDGF family that may have utility for generating cells that can be used for treating heart disease including vascular endothelial cell growth factor (“VEGF”, sometimes also referred to as “vascular permeability factor, or “VPF”) and placental growth factor (“PLGF”). Tischer et al., Biochem. Biophys. Res. Comm., 165(3), 1198-1206 (1989) and Maglione et al., Proc. Natl. Acad. Sci. USA, 88, 9267-9271 (1991), respectively. Both VEGF and PLGF form disulfide-bonded dimers from the eight highly conserved cysteine residues that appear in the PDGF homologous region of each monomeric unit of these PDGF family members. Tischer et al. and Maglione et al., ibid. The receptors for VEGF and PLGF are also in the same receptor subfamily as the PDGF receptors. Consequently, these “newer” members of the PDGF family are thought to be potentially useful as therapeutic products in wound repair and, according to the invention can be used herein to generate cells useful for treating and preventing heart disease and other vascular conditions.

Using Peptides to Identify or Isolate Cells with Cardioplastic Potential

The inventors have recently demonstrated that transplantation of bone marrow cells from young, but not older, adult mice can provide prolonged restoration of cardioprotective platelet-derived growth factor (PDGF) pathways in the aging host, which can reduce the extent of myocardial injury after acute coronary occlusion. Edelberg J M, et al. Circulation Research. 2002;90:E89-E93; Edelberg J M, et al. Circulation. 2002;105:608-613. The young cells populate the bone marrow of intact, non-irradiated senescent mice. Endothelial precursor cells (EPCs) derived from these transplanted young bone marrow cells are recruited to sites of angiogenesis in cardiac transplantation and myocardial infarction studies.

According to the present invention, the peptides of the invention can be used to help identify and isolate cells with cardioplastic potential that can be used to restore senescent bone marrow-mediated function and to protect the aging heart from myocardial injury. Cells with cardioplastic potential that are useful for regenerating aging heart functions include cardiac myocytes, cardiac endothelial cells, endothelial precursor cells, bone marrow cells or stem cells. Such cells can be identified and/or isolated by observing and collecting cells that bind to a peptide having SEQ ID NO:1.

Cells that bind peptides of the invention can be purified from preparations of bone marrow or circulating blood, using available methods such as cell sorting procedures, affinity chromatography, fluorescence-activated cell sorting (FACS) or immunomagnetic separation (for example, see Peichev et al., Blood, 2000, 95(3):952-958); and Otani et al., Nature Medicine, 2002, 8(9): 1004-1010, the contents of both of which are incorporated herein by reference in their entirety). For example, the purification procedure can lead at least to a two-fold, three-fold, five-fold, ten-fold, fifteen- fold, twenty-fold, or twenty-five fold increase in cells that bind the peptides of the invention (e.g., endothelial precursor cells) over the total population. The purified population of cells can contain at least 15%, at least 20%, at least 25%, at least 35%, or at least 50% of such cells.

The methods of the invention can also utilize cellular mixtures comprising 30%, 50%, 75%, 80%, 85%, 90% or 95% of cells that bind the peptides of the invention. The methods of the invention can also utilize cell mixtures comprising 99%, 99.9% and even 100% of such cells. Accordingly, cell populations utilized in the invention contain significantly higher levels of cells that bind the peptides of the invention than most populations of cells that exist in nature.

A population of cells that bind the peptide(s) of the invention can thus be isolated from bone marrow. Isolated cells are not necessarily pure cells; instead, isolated cells are removed from their natural source, environment or from the mammal where they naturally arose.

In some embodiments, cardiac myocytes are preferably used for regenerating aging heart tissues. Such cardiac myocytes can readily be generated by treating the cells that bind peptides of the invention (e.g. cells with cardioplastic potential, including stem cells and endothelial precursor cells) with appropriate factors and cytokines. For example, cardiac myocytes can be obtained from totipotent or pluripotent stem cells that are induced to differentiate into hematopoietic stem cells, which in turn can differentiate into endothelial precursor cells, which in turn can differentiate into cardiac myocytes.

Factors and cytokines useful for generating cardiac myocytes from cardiac endothelial cells, endothelial precursor cells, bone marrow cells or stem cells include platelet derived growth factor, vascular endothelial growth factor (VEGF), fibroblast growth factor-2 (FGF-2), Tenascin-C, peptides having SEQ ID NO: 1 and other factors.

For example, to promote cardiac myocytes formation from bone marrow cells, bone marrow cells can be obtained, cells that bind to the peptides of the invention can be isolated, and then those isolated cells can be cultured in a sufficient amount of platelet derived growth factor AB for a time and under conditions sufficient to generate myocytes. Platelet derived growth factor is commercially available and can be obtained, for example, from R&D Systems.

A sufficient amount of platelet derived growth factor AB is about 0.001 ng/mL to about 10 mg/mL, or about 0.01 ng/mL to about 1 mg/mL, or about 0.1 ng/mL to about 100 ng/mL or about 1 ng/mL to about 100 ng/mL platelet derived growth factor AB. In certain embodiments, cells isolated from bone marrow have been successfully treated with platelet derived growth factor AB at concentrations of about 10 ng/mL and 100 ng/mL.

The time used to generate cardiac myocytes from bone marrow by PDGF AB treatment can vary. For example, culturing bone marrow cells in the presence of PDGF AB for a time period of a few days (about 3 days) to several weeks (about 5 weeks) can lead to cardiac myocytes generation from bone marrow cells. In experiments described herein, bone marrow cells were successfully cultured for about 1 week in order to facilitate cardiac myocyte formation.

Conditions required for culturing bone marrow-derived cells to generate cardiac myocytes comprise the conditions normally employed for culturing mammalian cells in vitro. Inclusion of vascular endothelial growth factor (VEGF, at about 10 ng/mL), fibroblast growth factor-2 (FGF-2) (at about 5 ng/mL) and heparin (at about 50 μg/mL) also helps support the generation of cardiac myocytes from bone marrow cells in vitro.

Hence, the invention provides a method for generating cardiac myocytes from cells with cardioplastic potential (such as cardiac endothelial cells, endothelial precursor cells, bone marrow cells or stem cells) by isolating bone marrow cells from a human, exposing the bone marrow cells to a peptide comprising SEQ ID NO:1, isolating cells that bind to the peptide to form a pool of isolated cells, culturing the pool of isolated cells in the presence of a factor to generate a population of cardiac myocytes. These cardiac myocytes can be administered to a patient in a therapeutically effective amount to treat and/or prevent heart disease or other vascular conditions.

Peptides Conjugated to Reporter Molecules

According to the invention, peptides isolated or identified as described herein can be attached or conjugated to any known reporter molecule or other label or signaling agent. While the peptides of the invention have utility for identifying the location of, and for imaging vascular tissue, the invention is not limited to imaging just vascular tissues. The peptides of the invention can be used to detect, identify, locate and/or image any target molecule to which a peptide of the invention can bind, either in vitro or in vivo.

In one embodiment, the peptides and methods provided herein can be used to diagnose the location, extent, and pathologic composition of a vascular condition in the heart, or to assess whether bone marrow in or from a particular patient has a useful population of cardiac endothelial cells, young endothelial precursor cells, young bone marrow cells or stem cells. For example, detection of a peptide-conjugate capable of binding to heart tissue can provide information regarding the age, relative functioning, and regenerative potential of heart tissue or of a population of cells. The amount or extent of peptide binding can be used to diagnose the age, staging or severity of cardiac injury and the potential risk of thrombosis. Any reporter molecule, label or signaling agent known to one of skill in the art can be attached to the peptides of the invention as well as any and all agents used as diagnostic tools or to enhance diagnostic tools. Such peptide-conjugates can then be used in vivo or in vitro to image, locate or otherwise detect the biomolecule or tissue to which the peptide binds.

The peptide-conjugates of the invention can serve as a signal enhancing agent for medical diagnostic imaging, for example, for MRI, ultrasound, infrared and other imaging procedures. Peptide conjugates used for MRI, and radiodiagnostic imaging can, for example, have one or more amino acid side chains or linkers that are attached to chelating moieties, contrast agents or liposomes, such as unilamellar gadolinium-liposomes, manganese-liposomes, and iron-DTPA-stearate-liposomes.

One of skill in the art can conjugate such reporter molecules, labels and signaling agents to the present peptides using known techniques. For example, the followings references provide guidance on conjugation and use of such reporter molecules, labels and signaling agents in various diagnostic imaging procedures.

Bacic, G., M. R. Niesman, et al. (1990). “NMR and ESR study of liposome delivery of Mn2+ to murine liver.” Magn Reson Med 13(1): 44-61.

Bartolozzi, C., F. Donati, et al. (2000). “MnDPDP-enhanced MRI vs dual-phase spiral CT in the detection of hepatocellular carcinoma in cirrhosis.” Eur Radiol 10(11): 1697-702.

Bockhorst, K., M. Hoehn-Berlage, et al. (1993). “NMR-contrast enhancement of experimental brain tumors with MnTPPS: qualitative evaluation by in vivo relaxometry.” Magn Reson Imaging 11(5): 655-63.

Colet, J. M., L. Vander Elst, et al. (1998). “Dynamic evaluation of the hepatic uptake and clearance of manganese-based MRI contrast agents: a 31P NMR study on the isolated and perfused rat liver.” J Magn Reson Imaging 8(3): 663-9.

Diehl, S. J., K. J. Lehmann, et al. (1999). “MR imaging of pancreatic lesions. Comparison of manganese-DPDP and gadolinium chelate.” Invest Radiol 34(9): 589-95.

Fiel, R., E. Mark, et al. (1993). “Tumor-selective contrast enhancing agent, Mn(III)meso-[tri(4-sulfonatophenyl)phenyl]porphine (MnTPPS3).” Magn Reson Imaging 11(7): 1079-81.

Kim, S. W. and T. Kozuka (1990). “[Mn-TPPS4; a potential MRI contrast agent for localizing the normal aortic wall in rabbits].” Nippon Igaku Hoshasen Gakkai Zasshi 50(2): 192-94.

Laniado, M. and A. F. Kopp (1997). “[Current status of the clinical development of MR contrast media].” Rofo Fortschr Geb Rontgenstr Neuen Bildgeb Verfahr 167(6): 541-50.

Marchal, G., X. Zhang, et al. (1993). “Comparison between Gd-DTPA, Gd-EOB-DTPA, and Mn-DPDP in induced HCC in rats: a correlation study of MR imaging, microangiography, and histology.” Magn Reson Imaging 11(5): 665-74.

Maurer, J., A. Strauss, et al. (2000). “Contrast-enhanced high resolution magnetic resonance imaging of pigmented malignant melanoma using Mn-TPPS4 and Gd-DTPA: experimental results.” Melanoma Res 10(1): 40-6.

Maurer, J., A. Strauss, et al. (1999). “[Mn-TPPS4 in the diagnosis of malignant skin tumors. In vivo studies with high resolution magnetic resonance tomography in melanotic melanoma].” Radiologe 39(5): 422-7.

Navon, G., R. Panigel, et al. (1986). “Liposomes containing paramagnetic macromolecules as MRI contrast agents.” Magn Reson Med 3(6): 876-80.

Ni, Y., G. Marchal, et al. (1994). “Prolonged positive contrast enhancement with Gd-EOB-DTPA in experimental liver tumors: potential value in tissue characterization.” J Magn Reson Imaging 4(3): 355-63.

Plowchalk, D. R., J. P. Jordan, et al. (1987). “Effects of manganese (Mn++) and iron (Fe+++) on magnetic resonance imaging (MRI) characteristics of human placenta and amniotic fluid.” Physiol Chem Phys Med NMR 19(1): 35-41.

Rofsky, N. M. and J. C. Weinreb (1992). “Manganese (II) N,N′-dipyridoxylethylene-diamine-N,N′-diacetate 5,5′-bis(phosphate): clinical experience with a new contrast agent.” Magn Reson Q 8(3): 156-68.

Runge, V. M. (2000). “Safety of approved MR contrast media for intravenous injection.” J Magn Reson Imaging 12(2): 205-13.

Saeed, M., S. Wagner, et al. (1989). “Occlusive and reperfused myocardial infarcts: differentiation with Mn-DPDP—enhanced MR imaging.” Radiology 172(1): 59-64.

Schmiedl, U. P., J. A. Nelson, et al. (1992). “Hepatic contrast-enhancing properties of manganese-mesoporphyrin and manganese-TPPS4. A comparative magnetic resonance imaging study in rats.” Invest Radiol 27(7): 536-42.

Schwendener, R. A., R. Wuthrich, et al. (1990). “A pharmacokinetic and MRI study of unilamellar gadolinium-, manganese-, and iron-DTPA-stearate liposomes as organ-specific contrast agents.” Invest Radiol 25(8): 922-32.

Wang, C. (1998). “Mangafodipir trisodium (MnDPDP)-enhanced magnetic resonance imaging of the liver and pancreas.” Acta Radiol Suppl 415: 1-31.

Wilmes, L. J., M. Hoehn-Berlage, et al. (1993). “In vivo relaxometry of three brain tumors in the rat: effect of Mn-TPPS, a tumor-selective contrast agent.” J Magn Reson Imaging 3(1): 5-12.

Wolf, G. L., K. R. Burnett, et al. (1985). “Contrast agents for magnetic resonance imaging.” Magn Reson Annu: 231-66.

Wyttenbach, R., M. Saeed, et al. (1999). “Detection of acute myocardial ischemia using first-pass dynamics of MNDPDP on inversion recovery echoplanar imaging.” J Magn Reson Imaging 9(2): 209-14.

Yamamoto, T., A. Matsumura, et al. (1998). “Manganese-metalloporphyrin (ATN-10) as a tumor-localizing agent: magnetic resonance imaging and inductively coupled plasma atomic emission spectroscopy study with experimental brain tumors.” Neurosurgery 42(6): 1332-7; discussion 1337-8.

The present peptide-conjugates can also be used for ultrasound imaging. For example, the peptide can be grafted to the surface of a liposome that contains gas through conjugation of the peptide to a PEGylated lipid. The microbubbles so formed serve as signal enhancing agents for the ultrasound detection and imaging procedure. Such liposomes are described in U.S. Pat. No. 6,139,819 to Unger et al.

Useful chelating moieties tightly bind metal ions such as technetium-99m and indium-111. One of skill in the art can employ known procedures to make such technetium-99m and indium-111 labeled peptides for diagnostic imaging. See, for example, U.S. Pat. No. 6,107,459 to Dean. Peptide conjugates with radionuclides are also useful for therapy, including radiotherapy.

The peptides of the invention can also be conjugated with any available dye or fluorescent moiety or intermediate such as biotin. Such peptide-dye conjugates can, for example, be used with infrared spectroscopy to detect and locate the biomolecules or tissues to which the peptide can bind.

Therapeutic Agents

The invention also contemplates conjugating peptides to any therapeutic agent available to one of skill in the art. In the present context “a therapeutic agent” is also intended to comprise active metabolites and prodrugs thereof. An active “metabolite” is an active derivative of a therapeutic agent produced when the therapeutic agent is metabolised. A “prodrug” is a compound that is either metabolised to a therapeutic agent or is metabolised to an active metabolite(s) of a therapeutic agent. This invention can be used to administer therapeutic agents such as small molecular weight compounds, radionuclides, drugs, enzymes, peptides and/or proteins with biological activity, nucleic acids or genes that encode therapeutic polypeptides, expression vectors or other nucleic acid constructs, for example, naked plasmid DNAs, any vector carrying one or more genes, any sense or antisense RNA, any ribozyme, or any antibody.

For example, the peptides of the invention can be used to deliver platelet derived growth factor. Other examples of therapeutic agents that can be delivered include fusion proteins or fibrinolytic agents. Such therapeutic agents include, for example, thrombolytic agents such as streptokinase, tissue plasminogen activator, plasmin and urokinase, anti-thrombotic agents such as tissue factor protease inhibitors (TFPI), anti-inflammatory agents, metalloproteinase inhibitors, nematode-extracted anticoagulant proteins (NAPs) and the like. Liposomes can be used to facilitate delivery of such agents, for example, thrombolytic agents such as streptokinase, tissue plasminogen activator, plasmin and urokinase, anti-thrombotic agents such as tissue factor protease inhibitors (TFPI), anti-inflammatory agents, metalloproteinase inhibitors, nematode-extracted anticoagulant proteins (NAPs) and the like.

The peptides of the invention can be linked to such proteins or polypeptides. Upon administration, these therapeutic agents will become localized to sites of bone marrow or regenerating heart tissue and will help control, diminish or otherwise facilitate improved arterial blood flow in the region of an injury, atherosclerotic lesion or other site. The peptides of the invention can also be used to deliver nanoparticles, such as vectors for gene therapies (in a manner similar to the phage particles used for isolation of the peptides), as well as liposomes containing therapeutic agents like those listed herein.

Examples of therapeutic agents that can be linked to the peptides of the invention include the following:

1) Agents that control and modulate lipid levels (for example, HMG-CoA reductase inhibitors, thyromimetics, fibrates, agonists of peroxisome proliferator-activated receptors (PPAR) (including PPAR-alpha, PPAR-gamma and/or PPAR-delta);

2) Agents that control and modulate oxidative processes such as modifiers of reactive oxygen species or treatments that modify the production and/or activity of modified lipoproteins;

3) Agents that control and modulate insulin resistance and/or activity or glucose metabolism or activity including, but not limited to, agonists of PPAR-alpha, PPAR-gamma and/or PPAR-delta, modifiers of DPP-IV, and modifiers of glucocorticoid receptors;

4) Agents that control and modulate expression of receptors or adhesion molecules or integrins on endothelial cells or smooth muscle cells in any vascular location;

5) Agents that control and modulate the activity of endothelial cells or smooth muscle cells in any vascular location;

6) Agents that control and modulate inflammation associated receptors including, but not limited to chemokine receptors, RAGE, toll-like receptors, angiotensin receptors, TGF receptors, interleukin receptors, TNF receptors, C-reactive protein receptors, and other receptors involved in inflammatory signaling pathways including the activation of NF-kb;

7) Agents that control and modulate proliferation, apoptosis or necrosis of endothelial cells , vascular smooth muscle or lymphocytes, monocytes, and neutrophils adhering to or within the vessel;

8) Agents that control and modulate production, degradation, or cross-linking of any extracellular matrix proteins including, but not limited to, collagen, elastin, and proteoglycans;

9) Agents that control and modulate activation, secretion or lipid loading of any cell type within mammalian vessels;

10) Agents that control and modulate the activation, proliferation or any other modification of dendritic cells within mammalian vessels; and

11) Agents that control and modulate the activation, adhesion, or other processes that modify platelet events at the level of the vessel wall.

Such a therapeutic agent can be directly attached to a peptide of the invention, or it can be present in a phage particle, liposome or transformation vector available to one of skill in the art.

Peptides as Anti-Cell Migration Agents

According to the invention, tenascin-C can modulate cell migration and peptides or antibodies that bind tenascin-C can modulate such cell migration. Examples of peptides useful for modulating cell migration include peptides having SEQ ID NO:1. In some embodiments, the peptides and antibodies of the invention can inhibit cell migration. Thus, the invention contemplates methods of inhibiting cell migration, cancerous cell migration and treating cancer by administering the peptides and antibodies of the invention.

The peptides of the invention can be tested in appropriate animal models to further define the dosages for modulating cell migration. For example, the compounds of the invention can be tested in animals with known tumors, or animals that have been injected with tumor cells into a localized area. The degree or number of secondary tumors that form over time is a measure of metastasis and the ability of the compounds to inhibit such metastasis can be evaluated relative to control animals that have the primary tumor but receive no test compounds.

Accordingly peptides and antibodies of the invention are useful as therapeutic agents for inhibition of cell migration and treatment of metastatic cancer. More specifically, the peptide and antibody compositions and methods of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myclogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

Additionally, tenascin-C and cells that express tenascin-C are useful as tools for identifying further compounds, peptides and antibodies that can bind to tenascin-C and/or modulate cell migration. Thus, the invention provides a method of identifying an agent that can bind to tenascin-C that includes contacting a test agent with a tenascin-C polypeptide and observing whether the test agent binds to the tenascin-C polypeptide. In another embodiment, the invention provides a method for identifying agents that can modulate cell migration that includes contacting a first cell that expresses tenascin-C with a test agent and observing whether the cell migration of the first cell is altered relative to the cell migration of a second cell that expresses tenascin-C but was not contacted with the test agent.

Anti-Tenascin-C Antibodies

The invention provides antibodies that bind to tenascin-C polypeptides, for example, tenascin-C polypeptides having an amino acid sequence as set forth in SEQ ID NO:2 or a fragment of SEQ ID NO: 2, or conservative variants thereof. Such antibodies are useful for the diagnosis, immunization against, and treatment of cancer.

Antibodies can be prepared using an intact polypeptide or peptide fragment of interest as the immunizing antigen. The polypeptide or fragment used to immunize an animal can be derived from translated cDNA or chemical synthesis. A polypeptide or peptide fragment can be coupled to a carrier protein, if desired. Such commonly used carrier proteins which are chemically coupled to the peptide include keyhole limpet hemocyanin (KLH), thyroglobulin, bovine serum albumin (BSA), and tetanus toxoid. A coupled protein can be used to immunize the animal (e.g., a mouse, a rat, or a rabbit).

If desired, polyclonal or monoclonal antibodies can be further purified, for example, by binding to and elution from a matrix to which the polypeptide or peptide fragment to which the antibodies were raised is bound. Those of skill in the art will know of various techniques common in the immunology arts for purification and/or concentration of polyclonal antibodies, as well as monoclonal antibodies (Coligan, et al., Unit 9, Current Protocols in Immunology, Wiley Interscience, 1991, incorporated by reference).

It is also possible to use the anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

An antibody suitable for binding to a polypeptide or peptide fragment is specific for at least one portion of a region of the polypeptide. For example, one of skill in the art can use a peptide fragment to generate appropriate antibodies of the invention. Antibodies of the invention include polyclonal antibodies, monoclonal antibodies, and fragments of polyclonal and monoclonal antibodies.

The preparation of polyclonal antibodies is well-known to those skilled in the art (Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, section 2.4.1 (1992), which are hereby incorporated by reference). For example, a polypeptide or peptide fragment is injected into an animal host, preferably according to a predetermined schedule incorporating one or more booster immunizations, and the animal is bled periodically. Polyclonal antibodies specific for the polypeptide or peptide fragment may then be purified from such antisera by, for example, affinity chromatography using the polypeptide or peptide fragment coupled to a suitable solid support.

The preparation of monoclonal antibodies likewise is conventional (Kohler & Milstein, Nature, 256:495 (1975); Coligan et al., sections 2.5.1-2.6.7; and Harlow et al., Antibodies: A Laboratory Manual, page 726 (Cold Spring Harbor Pub. 1988)), which are hereby incorporated by reference. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (Coligan et al., sections 2.7.1-2.7.12 and sections 2.9.1-2.9.3; Barnes et al., Purification of Immunoglobulin G (IgG), in Methods in Molecular Biology, Vol. 10, pages 79-104 (Humana Press 1992)). Methods of in vitro and in vivo multiplication of monoclonal antibodies are available to those skilled in the art. Multiplication in vitro may be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an air reactor, in a continuous stirrer reactor, or immobilized or entrapped cell culture. Multiplication in vivo may be carried out by injecting cell clones into mammals histocompatible with the parent cells, e.g., osyngeneic mice, to cause growth of antibody-producing tumors. Optionally, the animals are primed with a hydrocarbon, especially oils such as pristine tetramethylpentadecane prior to injection. After one to three weeks, the desired monoclonal antibody is recovered from the body fluid of the animal.

Antibodies can also be prepared through use of phage display techniques. In one example, an organism is immunized with an antigen, such as a polypeptide or peptide fragment of the invention. Lymphocytes are isolated from the spleen of the immunized organism. Total RNA is isolated from the splenocytes and mRNA contained within the total RNA is reverse transcribed into complementary deoxyribonucleic acid (cDNA). The cDNA encoding the variable regions of the light and heavy chains of the immunoglobulin is amplified by polymerase chain reaction (PCR). To generate a single chain fragment variable (scFV) antibody, the light and heavy chain amplification products may be linked by splice overlap extension PCR to generate a complete sequence and ligated into a suitable vector. E. coli are then transformed with the vector encoding the scFV, and are infected with helper phage, to produce phage particles that display the antibody on their surface. Alternatively, to generate a complete antigen binding fragment (Fab), the heavy chain amplification product can be fused with a nucleic acid sequence encoding a phage coat protein, and the light chain amplification product can be cloned into a suitable vector. E. coli cells expressing the heavy chain fused to a phage coat protein are transformed with the vector encoding the light chain amplification product. The disulfide linkage between the light and heavy chains is established in the periplasm of E. coli. The result of this procedure is to produce an antibody library with up to 10⁹ clones. The size of the library can be increased to 10¹⁸ phages by later addition of the immune responses of additional immunized organisms that may be from the same or different hosts. Antibodies that recognize a specific antigen can be selected through panning. Briefly, an entire antibody library can be exposed to an immobilized antigen against which antibodies are desired. Phage that do not express an antibody that binds to the antigen are washed away. Phage that express the desired antibodies are immobilized on the antigen. These phage are then eluted and again amplified in E. coli. This process can be repeated to enrich the population of phage that express antibodies that specifically bind to the antigen. After phage are isolated that express an antibody that binds to an antigen, a vector containing the coding sequences for the antibody can be isolated from the phage particles and the coding sequences can be recloned into a suitable vector to produce an antibody in soluble form.

In another example, a human phage library can be used to select for antibodies, such as monoclonal antibodies, that bind to tenascin-C. Briefly, splenocytes may be isolated from a human and used to create a human phage library according to methods as described above and known in the art. These methods may be used to obtain human monoclonal antibodies that bind to tenascin-C. Phage display methods to isolate antigens and antibodies are known in the art and have been described (Gram et al., Proc. Natl. Acad. Sci., 89:3576 (1992); Kay et al., Phage display of peptides and proteins: A laboratory manual. San Diego: Academic Press (1996); Kermani et al., Hybrid, 14:323 (1995); Schmitz et al., Placenta, 21 Suppl. A:S106 (2000); Sanna et al., Proc. Natl. Acad. Sci., 92:6439 (1995)).

An antibody of the invention may be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described (Orlandi et al., Proc. Nat'l Acad. Sci. USA, 86:3833 (1989) which is hereby incorporated in its entirety by reference). Techniques for producing humanized monoclonal antibodies are described (Jones et al., Nature, 321:522 (1986); Riechmann et al., Nature, 332:323 (1988); Verhoeyen et al, Science, 239:1534 (1988); Carter et al., Proc. Nat'l Acad. Sci. USA, 89:4285 (1992); Sandhu, Crit. Rev. Biotech., 12:437 (1992); and Singer et al., J. Immunol., 150:2844 (1993), which are hereby incorporated by reference).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described (Green et al., Nature Genet., 7:13 (1994); Lonberg et al., Nature, 368:856 (1994); and Taylor et al., Int. Immunol., 6:579 (1994), which are hereby incorporated by reference).

Antibody fragments of the invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described (U.S. Pat. No. 4,036,945; 4,331,647; and 6,342,221, and references contained therein; Porter, Biochem. J., 73:119 (1959); Edelman et al., Methods in Enzymology, Vol. 1, page 422 (Academic Press 1967); and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Nat'l Acad. Sci. USA, 69:2659 (1972)). Alternatively, the variable chains can be linked by an intermolecular disulfide bond or cross-linked by chemicals such as glutaraldehyde (Sandhu, Crit. Rev. Biotech., 12:437 (1992)). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described (Whitlow et al., Methods: A Companion to Methods in Enzymology, Vol. 2, page 97 (1991); Bird et al., Science, 242:423 (1988), Ladner et al., U.S. Pat. No. 4,946,778; Pack et al., Bio/Technology, 11:1271 (1993); and Sandhu, Crit. Rev. Biotech., 12:437 (1992)).

Another form of an antibody fragment is a peptide that forms a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: A Companion to Methods in Enzymology, Vol. 2, page 106 (1991)).

An antibody of the invention may be coupled to a toxin. Such antibodies may be used to treat animals, including humans, that have cancer. For example, an antibody that binds to tenascin-C may be coupled to a tetanus toxin and administered to an animal suffering from cancer. The toxin-coupled antibody is thought to bind to a portion of a tenascin-C molecule presented on a cancerous cell, and then kill the cancerous cell.

An antibody of the invention may be coupled to a detectable tag. Such antibodies may be used within diagnostic assays to determine if an animal, such as a human, is suffering from cancer. Examples of detectable tags include, fluorescent proteins (i.e., green fluorescent protein, red fluorescent protein, yellow fluorescent protein), fluorescent markers (i.e., fluorescein isothiocyanate, rhodamine, texas red), radiolabels (i.e., ³H, ³²P, ¹²⁵I), enzymes (i.e., β-galactosidase, horseradish peroxidase, β-glucuronidase, alkaline phosphatase), or an affinity tag (i.e., avidin, biotin, streptavidin). Methods to couple antibodies to a detectable tag are known in the art. Harlow et al., Antibodies: A Laboratory Manual, page 319 (Cold Spring Harbor Pub. 1988).

The invention also provides peptide aptamers to a tenascin-C polypeptide. Peptide aptamers are peptides that bind to a tenascin polypeptide with affinities that are often comparable to those for monoclonal antibody-antigen complexes. In one example, aptamers can be isolated using the phage display libraries described herein or using a DNA library that contains a promoter, a start codon, a nucleic acid sequence coding for random peptides, and a nucleic acid sequence that codes for a histidine tag. This library is transcribed using a suitable polymerase, such as T7 RNA polymerase, after which a puromycin-containing poly A linker is ligated onto the 3′ end of the newly formed mRNAs. When these mRNAs are translated in vitro, the nascent peptides form covalent bonds to the puromycin of the linker to form an mRNA-peptide fusion molecule. The mRNA-peptide fusion molecules are then purified through use of Ni-NTA agarose and oligo-dT-cellulose. The mRNA portion of the fusion molecule is then reverse transcribed. The double-stranded DNA/RNA-peptide fusion molecules are then incubated with a tenascin-C polypeptide or peptide fragment and unbound fusion molecules are washed away. The bound fusion molecules are eluted from immobilized tenascin-C polypeptides and are then amplified by PCR. This process may be repeated to select for aptamers having high affinity for the tenascin-C polypeptides of the invention. The sequence of the nucleic acid coding for the aptamers can then be determined and cloned into a suitable vector. Methods for the preparation of peptide aptamers have been described (Wilson et al., Proc. Natl. Acad. Sci., 98:3750 (2001)). Accordingly, the invention provides aptamers that recognize tenascin-C polypeptides.

Compositions

Tenascin-C polypeptides, peptides that bind tenascin-C (e.g. peptide SEQ ID NO:1 or the aptamers described herein) and anti-tenascin-C antibodies can be formulated as pharmaceutical compositions. A pharmaceutical composition of the invention includes a tenascin-C polypeptide, or a peptide or antibody that binds tenascin-C in combination with a pharmaceutically acceptable carrier. The pharmaceutical compositions of the invention may be prepared in many forms that include tablets, hard or soft gelatin capsules, aqueous solutions, suspensions, and liposomes and other slow-release formulations, such as shaped polymeric gels.

The pharmaceutical compositions of the invention can also be coated onto, formulated into or placed within various devices, for example, controlled release devices, implantable tissue regenerating devices, biodegradable devices and devices that are implanted within tumor sites or within the vasculature or the heart. For example, stents, pacemakers and the like can be coated with tenascin-C. Alternatively, tenascin-C, peptides that bind tenascin-C or antibodies that bind tenascin-C can be incorporated into biodegradable devices for implantation at a variety of sites. For example, tenascin-C can be formulated into biodegradable stents that, when transplanted, slowly biodegrade and release tenascin-C. The tenascin-C encourages tissue re-modeling and vascularization of the cardiac site into which it was implanted, thereby regenerating injured or diseased cardiac tissues. Peptides and antibodies that bind tenascin-C can be formulated into implantable controlled release formulations that can be implanted at the site of an existing or a surgically removed tumor to prevent cell migration from that site.

An oral dosage form may be formulated such that the tenascin-C polypeptides, peptides, or antibodies are released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

Tenascin-C polypeptides, peptides, or antibodies can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the tenascin-C polypeptides, peptides, or antibodies may be in powder form, obtained by lyophilization from solution, for constitution with a suitable vehicle, e.g., sterile saline, before use.

Pharmaceutical compositions suitable for rectal administration can be prepared as unit dose suppositories. Suitable carriers include saline solution and other materials commonly used in the art.

For administration by inhalation, tenascin-C polypeptides, peptides, or antibodies can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, tenascin-C polypeptides, peptides, or antibodies may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, tenascin-C polypeptides, peptides, or antibodies may be administered via a liquid spray, such as via a plastic bottle atomizer.

Pharmaceutical compositions of the invention may also contain other ingredients such as flavorings, colorings, anti-microbial agents, or preservatives. It will be appreciated that the amount of a tenascin-C polypeptide, peptide, or antibody required for use in treatment will vary not only with the particular carrier selected but also with the route of administration, the nature of the condition being treated and the age and condition of the patient. Ultimately the attendant health care provider may determine proper dosage.

The invention is further illustrated by the following non-limiting Example.

### EXAMPLE

### Peptides that Bind Bone Marrow and Heart Microvascular Tissues

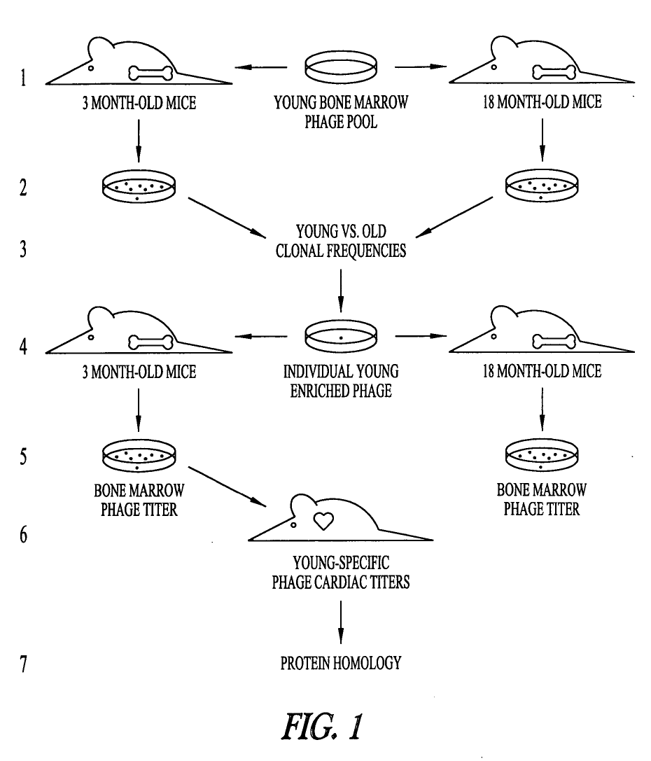

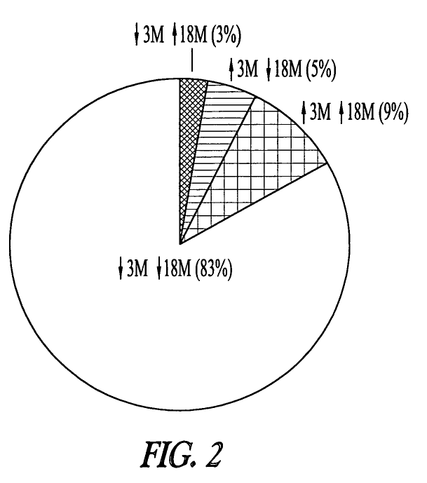

According to the invention, alterations in specific molecular epitopes of the aging bone marrow cells underlie the senescent impairment in the derivation of cardioprotective EPCs. In order to test this hypothesis, a functional genomic/proteomic approach was initiated. In particular, in vivo phage cyclic peptide libraries were used to identify age-associated changes in bone marrow receptors and adhesion molecules that contribute to the impairment in senescent cardiac vascular function. This approach is outlined in FIG. 1.

### Materials and Methods

Animals

Three-month-old and 18-month-old C57B1/6 mice and 4-month-old F344 rats were employed using procedures that complied with the Institutional Animal Care and Use Committee of Weill Medical College of Cornell University.

In Vivo Phage Biopanning

To identify young-specific bone marrow-homing phage, a phage library encoding a cyclically constrained seven amino acid variable region (New England Biolabs, Massachusetts) was injected into young (3 month) and old (18-month) adult mice, using methods described in Cai, D., et al., Age-associated impairment in TNF-alpha cardioprotection from myocardial infarction. Am J Physiol Heart Circ Physiol, 2003. 285(2): p. H463-69. Phage clones were selected for sequencing from phage pools eluted from young rather than old bone marrow. To confirm that these clones preferentially bound to young bone marrow, the clones were injected individually into young and old mice and young bone marrow-specific clones were re-selected from the young mouse's bone marrow. Young bone marrow-homing phage were then analyzed for cardiac binding to identify phage epitopes that preferentially bound to young cardiac vasculature as well as young bone marrow. The number of bound phage recovered per mg of tissue was quantified by serial dilution titration. The age-associated differential cardiac binding capacity of a candidate phage clone, ψR3Y32 was confirmed using methods described in Cai et al. (2004).

For immunohistochemical analysis of phage binding, hearts were harvested after phage injection, and then prepared for paraffin embedding and sectioning using standard histochemical procedures.

PDGF Injections

Cardiac injections of PDGF ligand were performed as described in Xaymardan et al., Senescent impairment in synergistic cytokine pathways that provide rapid cardioprotection in the rat heart. J Exp Med, 199(6): p. 797-804 (2004). Briefly, rats were anesthetized, an intercostal thoracotomy was performed and then the rats received intramyocardial injections of PDGF-AB (100 ng/50 μl PBS) at the anterior wall of the myocardium (two 25 μl injections, 2 mm apart) through a 28 gauge needle. The chest wall was closed, the lungs inflated and the rats were extubated. Twenty-four hours later, the rats were sacrificed and the hearts were harvested for paraffin embedding and sectioning, using standard techniques.

ROSA BM Transplantation

Bone marrow cells were isolated from young ROSA-26 mice as previously described in Xaymardan et al. (2004). Approximately 10⁷ bone marrow cells were injected via the tail vein into young irradiated mice. Twenty-eight days later, PDGF was injected intramyocardially as described above and 24 h later, mice were sacrificed for immunohistochemical analysis.

Immunohistochemistry

The following primary antibodies were used for immunostaining histological sections: anti-tenascin-C (Santa Cruz Biotechnology, California), anti-pIII (Mobitec, Germany), anti-β-galactosidase (Biogenesis, U.K.), anti-PDGFRα (Santa Cruz Biotechnology). For consistency, sections for all analyses were selected from the mid-papillary region of the ventricles. Primary antibodies were labeled with biotin and visualized with Texas Red- and FITC-conjugated avidin (Vector Labs, California).

For immunostaining of in vitro cultures, the following primary antibodies were used: anti-αv-integrin (Santa Cruz Biotechnology) and anti-Annexin II (Zymed Laboratories, California). Both antibodies were detected with FITC-conjugated secondary antibodies.

Cardiac Microvascular Endothelial Cell Isolation and In Vitro Adhesion and Migration Analysis

Rat cardiac microvascular endothelial cells were isolated from 3-month-old rats according to the method of Nishida et al. (1995). Cells were cultured on either collagen-coated or tenascin-C-coated (10 μg/ml) 8-well chamber slides (Nunc Nalge International, Illinois.) in minimal media (5% fetal bovine serum, 1% penicillin, 1% streptomycin in Dulbecco's modified Eagle's medium) for up to 48 hours and photographed daily. For cell adhesion analysis, media was removed from the wells 10 min or 3 h after plating and the number of cells that had failed to attach was counted using a hemocytometer. For migration analysis, cells were covered with 300 μl of collagen gel (Chemicon, California) after 3 h of culture on 10 μg/ml collagen or tenascin-C. The gel was allowed to polymerize for 1 hour at 37° C. and 200 μl of minimal media containing VEGF or PDGF (5 ng/ml) or no growth factor was added to the wells. Cells were incubated for up to 48 h, and analyzed for peak migration rates using an inverted microscope (Nikon, Japan): migratory distance of the 5 cells that had migrated furthest through the Z-axis of the gel was determined using the Z-plane focus knob of the microscope. Each experiment was repeated 4 times.

### Results

A phage biopan was performed with the aim of identifying epitopes that may be involved in the regulation of endothelial cell and/or EPC function in vascular remodeling. The procedures employed are outlined in FIG. 1. This strategy permitted isolation of phage clones that preferentially bound to young bone marrow and young cardiac vasculature, sites of EPC mobilization and cardiac angiogenesis, respectively. Over 300 bone marrow-enriched phage cyclic peptides were isolated and sequenced using these selection procedures. Age-specific bone marrow homing or binding of candidate clones were confirmed by in vivo biopanning with the individual phage, and quantified by phage titering.

FIG. 2 graphically illustrates the binding patterns of these 300 phage isolates in young (3 month old) and old (18 month old) mouse bone marrow. The pie chart provided in FIG. 2 shows to relative percentage of phage that exhibited preferential or increased (upward arrow) binding for young or old bone marrow as opposed to diminished or decreased (downward arrow) binding for young or old bone marrow. As illustrated, about 5% of the phage isolates preferentially bound to young bone marrow as opposed to old bone marrow. About 3% of the isolates preferentially bound to old bone marrow as opposed to young bone marrow.

A ψR3Y32 Phage Epitope Binds to Tenascin-C in the Cardiac Vasculature.

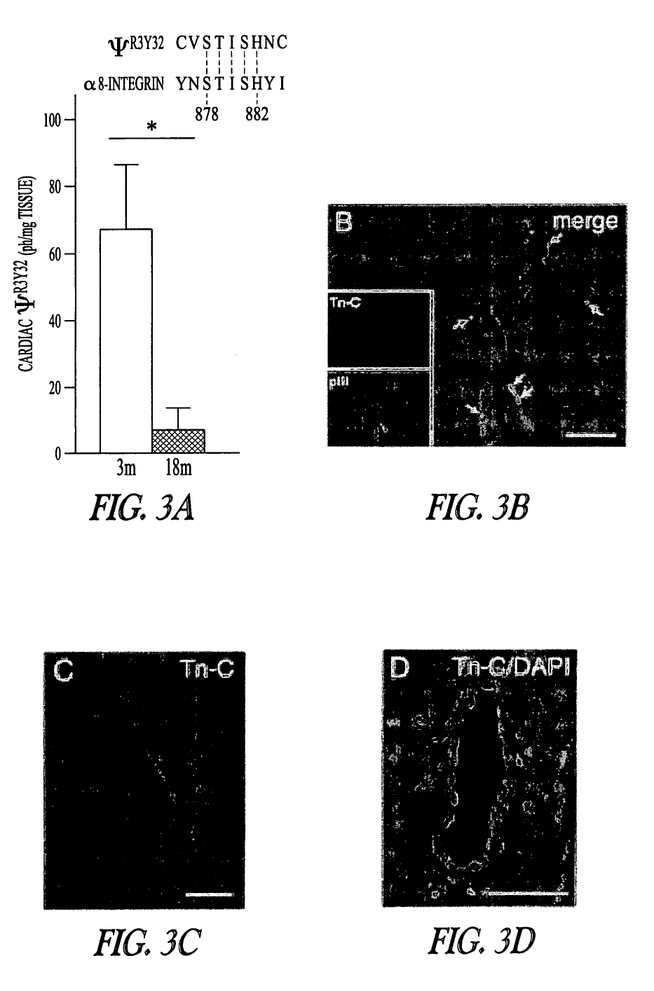

Three rounds of panning for phage that bound young cardiac vasculature resulted in enrichment of a phage clone, designated ψR3Y32. Individual injection of ψR3Y32 confirmed 10-fold higher levels of ψR3Y32 binding in young compared to old murine hearts (FIG. 3A). Injection of control, non-recombinant phage demonstrated negligible levels of binding within the heart (data not shown). These data indicate that binding partners for ψR3Y32 are preferentially expressed in the vasculature of young hearts.

The identity of polypeptides with structures like that of the ψR3Y32 phage was identified by homology searches performed using the BLAST software and databases available through the National Center for Biotechnology Information (http://www.ncbi.nlm.nih.gov/). The core peptide motif of ψR3Y32 (STISHN, SEQ ID NO:1) was found to be homologous to an extracellular region of alpha-8 integrin (Seq. Name#: ITGA8:aa878-882). Alpha-8 integrin is an extracellular matrix protein expressed by bone marrow stromal cells, and is critical for adult murine hematopoietic activity. Ohta M, et al. Blood. (1998) 91:4074-83. Given that ψR3Y32 mimics alpha-8 integrin binding, the specificity of ψR3Y32 binding to young tissues suggested that at least one binding partner for alpha-8 integrin may be diminished in senescent bone marrow and heart tissues.

Previous studies have demonstrated that tenascein-C (Tn-C) is a specific ligand for alpha-8 integrin. Schnapp L M, et al. J Biol Chem. (1995) 270:23196-202; Varnum-Finney B, et al. Neuron. (1995) 14:1213-22. Tenascin-C is involved in wound healing and tissue remodeling and is also upregulated after myocardial infarction. Mackie, E. J., W. Halfter, and D. Liverani, Induction of tenascin in healing wounds. J Cell Biol, 1988. 107(6 Pt 2): p. 2757-67; Jones, F. S. and P. L. Jones, The tenascin family of ECM glycoproteins: structure, function, and regulation during embryonic development and tissue remodeling. Dev Dyn, 2000. 218(2): p. 235-59; Imanaka-Yoshida K, et al. Lab Invest. (2001) 81:1015-24. Thus, tenascin likely has a role in vascular remodeling and cardioprotection.

To investigate whether tenascin is an in vivo receptor for ψR3Y32, expression of tenascin-C as well as its relationship to phage binding sites was analyzed in the rodent heart. Immunostaining for the phage coat protein, pill, confirmed the binding of ψR3Y32 to vessels within the heart (FIG. 3B). Tenascin-C expression was also observed primarily within the cardiac vasculature, most notably associated with venules, but also within the capillary network and in arterioles (FIGS. 3B-D). Tenascin-C expression was found to co-localize with pIII in the heart, suggesting that ψR3Y32 indeed mimics an endogenous binding partner for tenascin-C in the cardiac vasculature. Co-staining for tenascin-C and the nuclear marker 4,6-diamidino-2-phenylindole (DAPI) further confirmed that tenascin-C is expressed on the luminal surface of cardiac vessels, and therefore may act at sites of endothelial cell/EPC cell incorporation during vascular remodeling (FIG. 3D).

Endothelial Progenitor Cells Incorporate at Sites of Tenascin-C Expression

To examine whether endothelial cells and/or progenitor cells are indeed able to incorporate into the remodeling vasculature at sites of tenascin-C expression, bone marrow cells from ROSA-26 (β-galactosidase (β-gal)-positive) mice were injected into the tail veins of young mice, followed by intramyocardial injection of PDGF 28 days later. After 24 h, the mice were sacrificed and hearts examined histochemically.

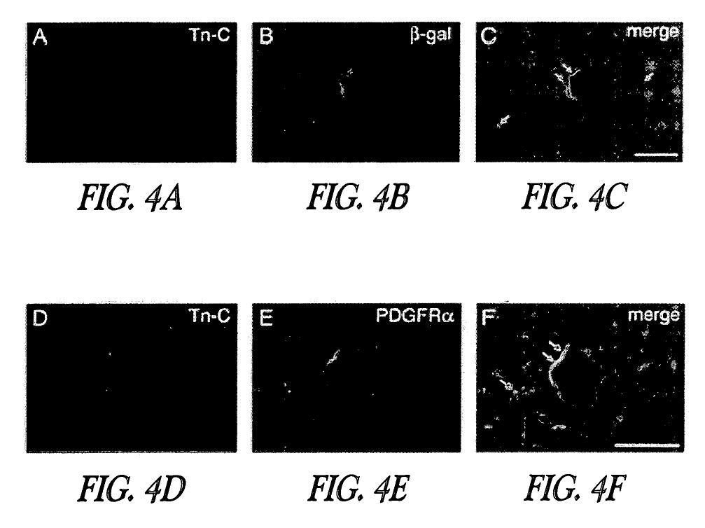

β-gal-positive, bone marrow-derived cells were detected at a number of sites within the vasculature, notably within venules and capillaries (FIG. 4A). Furthermore, the majority of these cells were localized to sites of tenascin-C expression (FIGS. 4B, C, see arrows in FIG. 4C), suggesting that tenascin-C may indeed mediate the incorporation of endothelial progenitor cells into the cardiac vasculature and/or regulate their maturation to an endothelial cell phenotype.

Tenascin-C expression was induced by PDGF during in vitro studies in rat aortic smooth muscle cells. In particular, a real-time PCR analysis of tenascin-C mRNA expression in cultured rat cardiac endothelial cells that were treated with platelet derived growth factor AB (PDGF-AB) indicated that tenascin-C expression increased in cardiac endothelial cells after administration of PDGF-AB (data not shown). Control cells received no PDGF. Cardiac endothelial cells from young (4-month old) and old (24-month old) rat hearts were tested. The number of treated and non-treated cultures tested was two (n=2 per group). Tenascin-C expression was higher in PDGF-treated cells and in younger (4 month old) as opposed to older (22 month old) cultures of rat cardiac endothelial cells.

The inventors have recently demonstrated that a sub-population of endothelial precursor cells express the PDGF receptor (PDGFR) PDGFRα, the major PDGFR involved in cardiac angiogenic mechanisms. Edelberg, J. M., et al., PDGF mediates cardiac microvascular communication. J Clin Invest, 1998. 102(4): p. 837-43. Furthermore, the inventors have shown that injection of PDGF ligand results in upregulation of PDGFRα expression in cardiac endothelial cells. Xaymardan, M., et al., Senescent impairment in synergistic cytokine pathways that provide rapid cardioprotection in the rat heart. J Exp Med, 2004. 199(6): p. 797-804. The localization of tenascin-C expression within the cardiac vasculature and induction of tenascin-C by PDGF in other vascular cells suggested that tenascin-C may play a role in PDGF-mediated angiogenic mechanisms.

As shown in FIGS. 4D and 4F, twenty-four hours after intramyocardial injection of PDGF-AB, tenascin-C protein expression was colocalized to sites of PDGFRα expression in the cardiac vasculature. Furthermore, immunohistochemical analysis of heart sections from rats treated with PDGF indicates that PDGF treatment increases tenascin-C expression (data not shown). Similarly, in vitro RT-PCR analysis of tenascin-C mRNA expression in cardiac endothelial cells cultured in the presence of exogenous PDGF ligand revealed an upregulation of tenascin-C, peaking at 6 h of PDGF treatment (data not shown). Thus, tenascin-C is upregulated by PDGF signaling and is associated with local cardiovascular PDGFRα expression.

Tenascin-C is Anti-Adhesive for Cardiac Endothelial Cells at Early, but not Late Time-Points

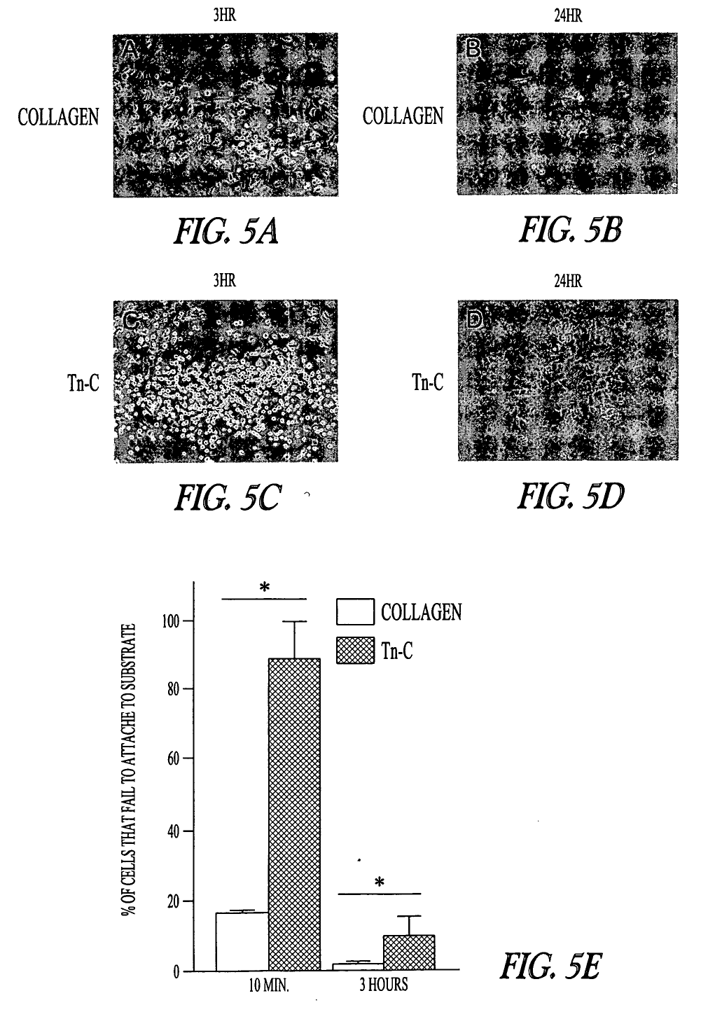

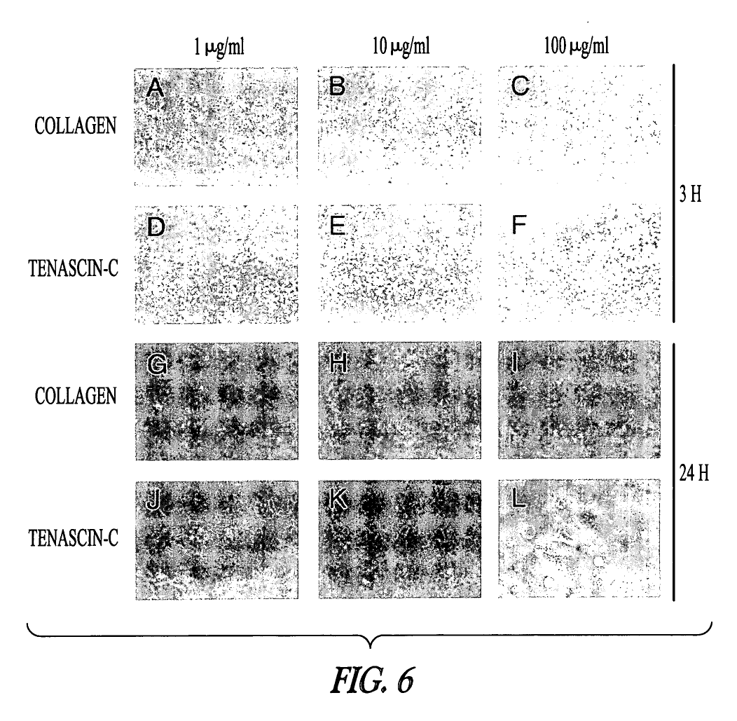

The role of tenascin-C in the regulation of cardiac angiogenesis, endothelial cell phenotype and behavior was further analyzed by observing whether tenascin-C influenced cell adhesion. Cardiac endothelial cells were isolated from young hearts and cultured on either tenascin-C or collagen at a concentration of 10 μg/ml. After 3 hours of incubation, cells cultured on collagen had already attached well and were beginning to spread (FIGS. 5A, B). Cells grown on tenascin-C, however, were rounded and poorly attached to the substrate at this time-point (FIGS. 5C, D). After 24 hours of culture, cells on tenascin-C resembled those cultured on collagen: they had become well attached and spread and formed a typical endothelial monolayer. Thus, it appeared that tenascin-C has a short-term anti-adhesive effect on cardiac endothelial cells. Indeed, within 10 min of plating, only 16.9% of cells have failed to attach to a collagen substrate, however, 86.9% of cells do not attach to tenascin-C within this time (FIG. 5E). After 3 h of culture, 99% of cells were attached to the collagen substrate, while approximately 10% of cells on tenascin-C still fail to adhere in this time. Thus, tenascin-C can mediate endothelial cell attachment but, at least within the first few hours of endothelial cell binding to tenascin-C, this attachment is weak and cells maintain a rounded phenotype.

The time period and substrate concentration effects upon cell attachment were further examined by plating rat microvascular endothelial cells on various concentrations (1 μg/ml, 10 μg/ml and 100 μg/ml) of collagen and tenascin-C and observing the attachment of cells to these substrates. As shown in FIGS. 6A-C, within 3 hr, cells on collagen were well attached and beginning to spread into a monolayer. In contrast, cells plated on tenascin-C for 3 hr remained rounded, especially when higher concentrations of tenascin-C were used (FIGS. 6D-F). By 24 hr, cells plated on lower concentrations of tenascin-C became well attached and had spread (FIGS. 6J-K), while those plated on the highest concentration of tenascin-C (100 μg/ml) formed multi-layered cell clusters (FIG. 6L).

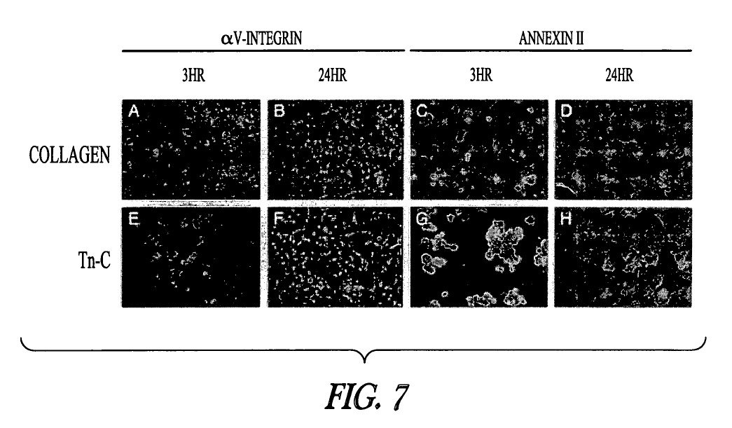

Tenascin-C is known to mediate attachment to various cell types via both integrin and non-integrin interactions. See, Jones, F. S. and P. L. Jones, The tenascin family of ECM glycoproteins: structure, function, and regulation during embryonic development and tissue remodeling. Dev Dyn, 2000. 218(2): p. 235-59 (review). Thus, experiments were performed to determine whether the anti-adhesive endothelial cell phenotype induced by tenascin-C is mediated by these integrin or other interactions. Immunohistochemical analysis revealed that αv-integrin was expressed by well-attached and spread cells and was localized to the cell-cell borders (FIGS. 7A, B, F). On rounded, poorly attached cells fewer cell-cell interactions exist, but αv-integrin nevertheless was also localized at the periphery of the cells (FIG. 7E).

In contrast, a more striking difference was observed in patterning of annexin II in attached cells compared to rounded non-attached cells. While well-attached cells expressed annexin II diffusely at relatively low levels throughout the cytoplasm (FIGS. 7C, D, H), rounded cells expressed annexin II at their periphery (FIG. 7G). This differential expression pattern suggests that annexin II may indeed mediate some of tenascin-C's anti-adhesive and anti-spreading function on cardiac endothelial cells.

Tenascin-C Promotes Cardiac Endothelial Cell Migration in Response to Angiogenic Growth Factors

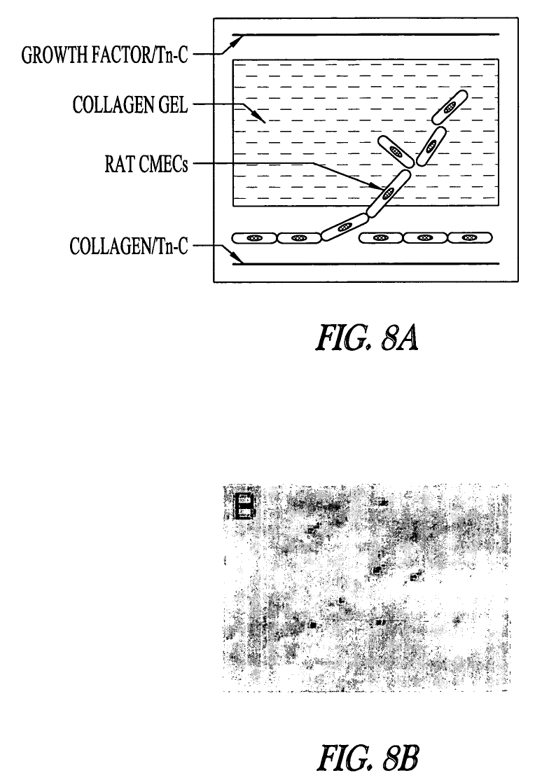

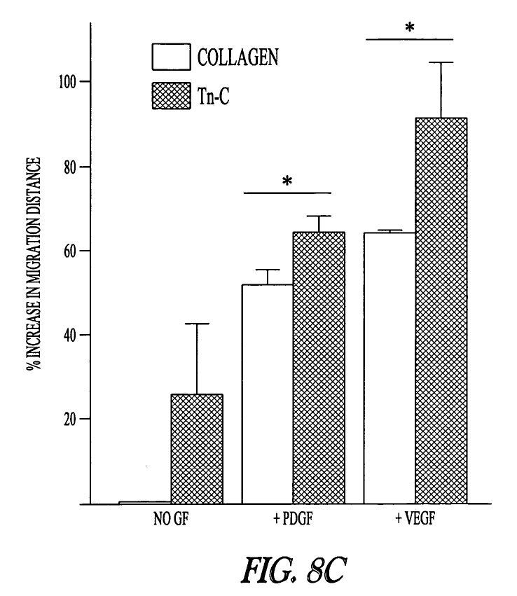

Based on the phenotype displayed by cardiac endothelial cells cultured on tenascin-C, experiments were performed to ascertain whether tenascin-C may act to maintain these cells in a rounded, poorly-adherent state, to promote accelerated migratory activity compared to those cells that attach well and spread onto a collagen substrate. A three-dimensional assay system was developed in which cardiac endothelial cells were cultured on either collagen or tenascin-C for 3 h, and then migration through a collagen gel was analyzed in response to PDGF, VEGF, or the absence of angiogenic growth factors (FIGS. 8A, B). After 48 hours, peak migration distances through the Z-axis of the gel were determined (FIG. 8C).

This analysis revealed that while cardiac endothelial cells were able to migrate on both tenascin-C and collagen in the absence of stimulatory growth factors, migration was greatly enhanced in the presence of either PDGF or VEGF. Peak migration distance was increased for cells cultured on collagen by 51.4% and 63.3% in response to PDGF and VEGF, respectively, compared to cells cultured on collagen in the absence of chemotactic growth factors.

Strikingly, cells cultured on tenascin-C had migration rates that were even further enhanced in response to these growth factors (64.3% and 91.6%, respectively), demonstrating that maintenance of cells in a poorly attached state on tenascin-C indeed results in promotion of migratory capacity in response to angiogenic signals. This in vitro analysis therefore indicates that tenascin-C acts at sites of vascular remodeling to promote angiogenic activity.

### REFERENCES

1. Edelberg J M, et al. Circulation Research. 2002;90:E89-E93.
2. Edelberg J M, et al. Circulation. 2002;105:608-613.
3. Schnapp L M, et al. J Biol Chem. 1995;270:23196-202.
4. Vamum-Finney B, et al. Neuron. 1995;14:1213-22.
5. Ohta M, et al. Blood. 1998;91 :4074-83.
6. Imanaka-Yoshida K, et al. Lab Invest. 2001;81:1015-24.
7. Matsumoto K, et al. Biochem Biophys Res Commun. 2002;290: 1220-7.
8. Seiffert M, et al. Matrix Biol. 1998;17:47-63.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A composition comprising a therapeutically effective amount of tenascin-C and a pharmaceutically acceptable carrier.

2. The composition of claim 1, wherein a therapeutically effective amount of tenascin is an amount that can modulate migration of endothelial cells.

3. The composition of claim 1, wherein the composition is formulated for controlled delivery of the tenascin-C.

4. The composition of claim 1, wherein the composition is formulated for delivery to a mammalian heart or mammalian vasculature.

5. The composition of claim 1, wherein the carrier is a device for insertion into a mammalian heart or mammalian vasculature.

6. The composition of claim 5, wherein the device is a stent or a cardiac pacemaker.

7. The composition of claim 6, wherein the device is biodegradable.

8. The composition of claim 1, wherein the tenascin is linked to an agent with binding affinity for biomolecules in the heart.

9. The composition of claim 8, wherein the agent is a peptide comprising SEQ ID NO:1 (STISHN).

10. A peptide comprising SEQ ID NO:1 (STISHN) that preferentially binds in vivo to mammalian cardiac endothelial cells.

11. The peptide of claim 10, wherein the peptide can also preferentially bind in vivo to mammalian bone marrow or Tenascin-C.

12. The peptide of claim 10, wherein the peptide is linked to a therapeutic agent.

13. The peptide of claim 12, wherein the therapeutic agent is platelet derived growth factor.

14. The peptide of claim 13, wherein the platelet derived growth factor comprises any one of SEQ ID NO:3-6.

15. A composition comprising a carrier and a therapeutically effective amount of the peptide of claim 10.

16. The composition of claim 15, where the composition is formulated for administration to a cancerous tissue.

17. An isolated nucleic acid encoding the peptide of claim 10.

18. An expression cassette comprising the nucleic acid of claim 17 and a promoter operable in a host cell.

19. The expression cassette of claim 18, wherein the host cell is Escherichia coli.

20. The expression cassette of claim 18, wherein the nucleic acid further encodes a therapeutic agent.

21. The expression cassette of claim 20, wherein the therapeutic agent is platelet derived growth factor.

22. The expression cassette of claim 21, wherein the platelet derived growth factor comprises any one of SEQ ID NO:3-6.

23. A host cell comprising a nucleic acid encoding the peptide of claim 10.

24. The host cell of claim 23, wherein the host cell is Escherichia coli.

25. The host cell of claim 23, wherein the nucleic acid further encodes a therapeutic agent.

26. The host cell of claim 25, wherein the therapeutic agent is platelet derived growth factor.

27. The host cell of claim 26, wherein the platelet derived growth factor comprises any one of SEQ ID NO:3-6.

28. A method for identifying endothelial precursor/progenitor cells in a cell population comprising contacting the cell population with a polypeptide comprising the peptide of claim 10, and identifying cells to which the peptide binds.

29. The method of claim 28, wherein the peptide further comprises a detectable label.

30. A method for isolating endothelial precursor/progenitor cells from a mixed cell population comprising contacting the mixed cell population with a polypeptide comprising the peptide of claim 10, and isolating the cells to which the peptide binds.

31. The method of claim 30, wherein the peptide further comprises a detectable label.

32. The method of claim 30, wherein the cells to which the peptide binds are isolated by cell sorting.

33. Endothelial cells and endothelial precursor/progenitor cells exhibiting cardioplastic potential that are isolated from a mixed cell population by a method comprising contacting the mixed cell population with a polypeptide comprising the peptide of claim 10, and isolating cells to which the peptide binds.

34. A method of restoring cardiac angiogenic function in a patient having diminished cardiac angiogenic function, said method comprising isolating bone marrow cells from a human, exposing the bone marrow cells to polypeptide comprising the peptide of claim 10, isolating cells that bind to the peptide to form a pool of isolated cells, culturing the pool of isolated cells in the presence of PDGF to generate a population of cardiac myocytes and administering to the patient a therapeutically effective amount of the cardiac myocytes.

35. The method of claim 34, wherein the pool of isolated cells are endothelial cells, endothelial precursor/progenitor cells, bone marrow cells or stem cells.

36. The method of claim 34, wherein the pool of isolated cells are also cultured in the presence of tenascin-C.

37. The method of claim 34, wherein the bone marrow cells are isolated from the patient.

38. A method of restoring cardiac angiogenic function in a patient having diminished cardiac angiogenic function, said method comprising administering to the patient a therapeutically effective amount of tenascin-C.

39. A method of restoring cardiac angiogenic function in a patient having diminished cardiac angiogenic function, said method comprising administering to the patient a therapeutically effective amount of the peptide of claim 10, wherein the peptide is linked to a therapeutic agent.

40. A method of restoring cardiac angiogenic function in a patient having diminished cardiac angiogenic function, said method comprising administering to the patient a therapeutically effective amount of the peptide of claim 10, wherein the peptide is linked to tenascin-C.

41. A method of restoring cardiac angiogenic function in a patient having diminished cardiac angiogenic function, said method comprising administering to the patient a therapeutically effective amount of the peptide of claim 10, wherein the peptide is linked to platelet derived growth factor.

42. A method of diagnosing impaired cardiac angiogenic function in a patient comprising administering a polypeptide comprising the peptide of claim 10 to the patient and detecting whether the peptide binds to cardiovascular tissues in the patient.

43. The method of claim 42, wherein the peptide is linked to a detectable label.

44. The method of claim 42, wherein detecting whether the peptide binds to cardiac tissues in the patient further comprises quantifying how much of the peptide binds to cardiac tissues in the patient.

45. A method of generating cardiac myocytes from cardiac endothelial cells, endothelial precursor/progenitor cells, bone marrow cells or stem cells comprising contacting the cardiac endothelial cells, endothelial precursor cells, bone marrow cells or stem cells with the peptide of claim 10 to generate a population of cardiac myocytes.

46. The method of claim 45, wherein the method further comprises contacting the cardiac endothelial cells, endothelial precursor cells, bone marrow cells or stem cells with platelet derived growth factor.

47. A method of generating cardiac myocytes from cardiac endothelial cells, endothelial precursor/progenitor cells, bone marrow cells or stem cells comprising contacting the cardiac endothelial cells, endothelial precursor/progenitor cells, bone marrow cells or stem cells with a tenascin-C to generate a population of cardiac myocytes.

48. The method of claim 47, wherein the method further comprises contacting the cardiac endothelial cells, endothelial precursor cells, bone marrow cells or stem cells with platelet derived growth factor.

49. A method of inhibiting cancer cell migration in a mammal comprising administering to the mammal a therapeutically effective amount of a polypeptide comprising the peptide of claim 10.

50. A method of generating pluripotent cells from cardiac endothelial cells, endothelial precursor/progenitor cells, or bone marrow cells comprising contacting the cardiac endothelial cells, endothelial precursor/ progenitor cells, or bone marrow cells with a tenascin-C to generate a population of pluripotent cells.


SEQUENCE LISTING (6 sequences)

SEQ ID NO: 1 - protein, 6 aa, Homo sapiens
STISHN

SEQ ID NO: 2 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGLDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDLTGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAQALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 3 - protein, 204 aa, Homo sapiens
MRTWACLLLLGCGYLAHALAEEAEIPRELIERLARSQIHSIRDLQRLLEIDSVGAEDALETNLRAHGSHTVKHVPEKRPVPIRRKRSIEEAIPAVCKTRTVIYEIPRSQVDPTSANFLIWPPCVEVKRCTGCCNTSSVKCQPSRVHHRSVKVAKVEYVRKKPKLKEVQVRLEEHLECACATSNLNPDHREEETGRRRESGKKRK

SEQ ID NO: 4 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 5 - protein, 161 aa, Homo sapiens
RSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 6 - protein, 119 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFL